(19) United States
(12) Patent Application Publication
(10) Pub. No.: US 2023/0404609 A1
(43) Pub. Date: Dec. 21, 2023

(54) Systems and Methods for Duodenal Mucosal Resection

(71) Applicant: Virender K. Sharma, Paradise Valley, AZ (US)

(72) Inventor: Virender K. Sharma, Paradise Valley, AZ (US)

(21) Appl. No.: 18/320,970

(22) Filed: May 20, 2023

Related U.S. Application Data
(60) Provisional application No. 63/365,079, filed on May 20, 2022.

(51) Int. Cl. A61B 17/32; A61B 17/3205

(52) U.S. Cl. CPC A61B 17/320016; A61B 17/32056; A61B 2017/00296

(57) ABSTRACT

Methods and systems are provided for an endoscopic tissue resection and or tissue removal device for use with an endoscope. The distal end of the endoscope includes the resection device with one or more elastic bands and shape memory material anchors associated with each band. Post-deployment of a band from the distal end of the endoscope, the associated shape memory material provides an anchor into the target mucosal tissue that the deployed band is positioned to constrict.

### CROSS-REFERENCE

The present application relies upon U.S. Patent Provisional Application No. 63/365,079, titled “Systems and Methods for Duodenal Mucosal Resection”, and filed on May 20, 2022, for priority. The above-mentioned application is herein incorporated by reference in its entirety.

### FIELD

The present specification is related generally to resection of portions of the gastrointestinal tract to treat gastrointestinal conditions. More specifically, the present specification is related to devices and methods for resecting portions of the proximal foregut to treat diabetes, obesity, metabolic syndrome, fatty liver, nonalcoholic steatohepatitis (NASH), nonalcoholic fatty liver disease (NAFLD), or polycystic ovarian syndrome (PCOS).

### BACKGROUND

Type 2 diabetes mellitus (T2D) is increasing at a disturbing rate throughout the world with an estimated global prevalence of 552 million by 2030. According to 2017 International Diabetes Federation (IDF) statistics, there are approximately 425 million people with diabetes worldwide. In the United States, there are an estimated 30.3 million adults living with diabetes, and its prevalence has been rising rapidly, with at least 1.5 million new diabetes cases diagnosed each year. Diabetes is a major public health epidemic despite recent advances in both pharmaceutical and technologic treatment options. The therapeutic goal of a glycated hemoglobin (HbA1c) level of ≤53 mmol/mol³ is achieved by less than half of the patients with T2D despite lifestyle interventions and an increasing number of medical treatment options. Bariatric surgery has proven to be successful in patients with class I, II and III obesity. In moderately obese patients with T2D, bariatric surgery is superior to intensive medical therapy alone. However, bariatric surgery is not a scalable solution for the growing T2D pandemic as most bariatric surgery procedures are invasive, irreversible and associated with some morbidity.

Fueled by increasing obesity rates, NAFLD has emerged as a leading global cause of chronic liver disease in the past few decades. The global prevalence of NAFLD among adults is estimated to be 23-25%. Up to 20% of people with NAFLD are affected by NASH. Despite growing prevalence, the factors influencing NAFLD development and subsequent progression to NASH, liver fibrosis, cirrhosis and hepatocellular carcinoma are poorly understood. In the vast majority of patients, NAFLD emerges in the context of metabolic syndrome, with insulin resistance an important pathophysiological mechanism. Liver cancer is now the second leading cause of years of life lost among all cancers globally.

Polycystic ovary syndrome (PCOS) is a hormonal disorder common among women of reproductive age. PCOS is now recognized as an important metabolic as well as reproductive disorder conferring substantially increased risk for type 2 diabetes. Affected women have marked insulin resistance, independent of obesity. Obese women with PCOS are insulin resistant. Early diagnosis and treatment along with weight loss may reduce the risk of long-term complications such as type 2 diabetes and heart disease.

The duodenum has become increasingly recognized as a metabolic signaling center that seems to play a role in regulating insulin action and, therefore, insulin resistance states. Hence, the duodenum has become a metabolic treatment target through bariatric surgery leanings and the specific observation that bypassing, excluding or altering duodenal nutrient exposure elicits favorable metabolic changes including immediate improvements in glycemic regulation after bariatric surgery, which do not appear to be due to malabsorption or the substantial weight loss often observed later post-surgery. Studies suggest a critical physiological and pathophysiological role of the small bowel in metabolic homeostasis. The easy endoscopic accessibility of the duodenum makes it a potential target for disease-modifying intervention for diseases associated with insulin resistance.

Two major mechanisms have been hypothesized to explain the rapid improvement of T2DM. Firstly, the foregut hypothesis suggests that improved glycemia after proximal intestinal exclusion results from reduced secretion of diabetogenic hormones/anti-incretin factors in response to the absence of nutrition in the proximal small intestine. For example, intestinal glucagon synthesis has been suggested to decrease after exclusion of the proximal intestine. Secondly, the hindgut hypothesis attributes improved glycemic control to enhanced secretion of incretins, like glucagon-like peptide-1 (GLP-1), in response to undigested nutrients in the distal small intestine. These theories are not mutually exclusive and additional factors likely play a role in the rapid glycemic improvement after bariatric surgery. Particularly, glucose dependent insulinotropic polypeptide (GIP), a gut hormone which stimulates glucagon secretion in response to a meal, may also be involved. Furthermore, caloric intake is of importance in improvement of T2DM.

The duodenum plays an important role in the control of glucose homeostasis through various mechanisms. A few key mechanisms include:

1. Enteroendocrine Hormone Release: The duodenum contains specialized cells called enteroendocrine cells, which secrete several hormones, including glucagon-like peptide-1 (GLP-1) and glucose-dependent insulinotropic peptide (GIP). When glucose enters the duodenum from the stomach, these hormones are released into the bloodstream. GLP-1 stimulates insulin secretion from pancreatic beta cells in a glucose-dependent manner. It also inhibits glucagon secretion from pancreatic alpha cells, slows down gastric emptying, and promotes satiety. GIP also stimulates insulin release from pancreatic beta cells in response to elevated blood glucose levels. It enhances nutrient uptake and storage by promoting the release of insulin and inhibiting glucagon secretion.

2. Neural Signaling: The duodenum communicates with the brain via the vagus nerve, which plays a crucial role in regulating glucose homeostasis. Sensory signals from the duodenum, including the presence of glucose, activate the vagal afferent neurons, which transmit information to the brain. The brain, in turn, sends signals to regulate pancreatic hormone secretion, hepatic glucose production, and other metabolic processes.

3. Incretin Effect: The “incretin effect” refers to the enhanced insulin secretion observed when glucose is ingested orally compared to intravenously. This effect is attributed to the duodenal release of GLP-1 and GIP in response to oral glucose intake. These incretin hormones potentiate glucose-stimulated insulin secretion, leading to better glucose clearance.

4. Intestinal Gluconeogenesis: The duodenum, along with the jejunum, is capable of gluconeogenesis, the production of glucose from non-carbohydrate precursors. This process can contribute to glucose homeostasis by providing a source of glucose during fasting or periods of low blood glucose.

Overall, the duodenum exerts control over glucose homeostasis through the release of enteroendocrine hormones, neural signaling, the incretin effect, and intestinal gluconeogenesis. These mechanisms work together to regulate pancreatic hormone secretion, insulin sensitivity, and hepatic glucose production, helping to maintain glucose levels within a normal range.

The duodenum, as the first segment of the small intestine, can influence the development and progression of fatty liver disease through several mechanisms. Mechanisms underlying the relationship between the duodenum and fatty liver disease are complex and not fully elucidated. Various factors, including genetics, diet, lifestyle, and underlying metabolic conditions, can interact with the duodenal processes mentioned below to influence the development and progression of fatty liver disease. The key mechanisms explaining how duodenal influence NAFLD or fatty liver disease include:

Bile Acid Metabolism: The duodenum is responsible for receiving bile acids from the gallbladder, which are important for the digestion and absorption of dietary fats. Bile acids aid in the emulsification and absorption of dietary lipids in the small intestine. Disruption of bile acid metabolism or impaired bile acid flow from the duodenum can contribute to the development of fatty liver disease. Alterations in bile acid composition and availability can impact lipid metabolism in the liver and contribute to the accumulation of fat.

Gut Microbiota: The duodenum and the rest of the small intestine host a diverse population of microorganisms collectively known as the gut microbiota. The gut microbiota plays a crucial role in various metabolic processes, including lipid metabolism. Disturbances in the gut microbiota composition, often referred to as dysbiosis, can impact the metabolism of dietary fats and contribute to the development of fatty liver disease.

Intestinal Inflammation and Barrier Dysfunction: Chronic low-grade inflammation and increased intestinal permeability (leaky gut) are associated with the pathogenesis of fatty liver disease. The duodenum, being a critical site of nutrient absorption, can be influenced by various dietary and environmental factors, leading to intestinal inflammation and disruption of the intestinal barrier. Increased intestinal permeability can allow the translocation of bacterial products, such as lipopolysaccharides (LPS), into the bloodstream, triggering systemic inflammation and promoting the development of liver steatosis.

Incretin Hormones: As mentioned earlier, the duodenum releases incretin hormones, such as GLP-1 and GIP, in response to nutrient ingestion. These hormones not only regulate glucose homeostasis but also have effects on lipid metabolism. GLP-1 has been shown to reduce hepatic fat accumulation and improve insulin sensitivity, potentially protecting against fatty liver disease. GIP may also influence lipid metabolism and contribute to the development of hepatic steatosis.

Nutrient Sensing and Signaling: The duodenum is a critical site for nutrient sensing and signaling, relaying information about nutrient availability to other organs, including the liver. Changes in nutrient composition and absorption in the duodenum can impact signaling pathways involved in lipid metabolism and influence the development of fatty liver disease.

The duodenal submucosa, the layer beneath the mucosal lining of the duodenum, plays a role in glucose homeostasis through several mechanisms. Some of the key mechanisms of action related to the duodenal submucosa include:

Enteroendocrine Hormone Secretion: The duodenal submucosa contains specialized enteroendocrine cells that secrete various hormones involved in glucose regulation. These include glucagon-like peptide-1 (GLP-1) and glucose-dependent insulinotropic peptide (GIP). When nutrients, especially glucose, encounter the duodenal submucosa, it triggers the release of these hormones into the bloodstream. GLP-1 promotes glucose-dependent insulin secretion from pancreatic beta cells. It also inhibits glucagon secretion from pancreatic alpha cells, slows down gastric emptying, and promotes satiety. GIP stimulates insulin release from pancreatic beta cells in response to elevated blood glucose levels. It enhances nutrient uptake and storage by promoting the release of insulin and inhibiting glucagon secretion.

Neural Signaling: The duodenal submucosa is innervated by a network of nerves, including the enteric nervous system (ENS) and autonomic nerves. These nerves sense and transmit signals related to glucose levels and participate in the regulation of glucose homeostasis. Sensory signals from the duodenal submucosa are transmitted via nerve pathways to the brain, which in turn modulates pancreatic hormone secretion, hepatic glucose production, and other metabolic processes.

Nutrient Sensing: The duodenal submucosa contains specialized nutrient-sensing cells, such as L-cells and K-cells. These cells can directly sense glucose and other nutrients in the intestinal lumen and respond by releasing hormones like GLP-1 and GIP, as mentioned earlier. This nutrient sensing helps regulate insulin secretion and other metabolic processes to maintain glucose homeostasis.

GLP-1 Receptor Signaling: GLP-1 released from the duodenal submucosa acts on target tissues throughout the body by binding to GLP-1 receptors. Activation of GLP-1 receptors has various effects that promote glucose homeostasis, such as stimulating insulin secretion, inhibiting glucagon secretion, enhancing glucose uptake by cells, and improving pancreatic beta cell function.

These mechanisms collectively contribute to the regulation of glucose homeostasis by the duodenal submucosa. By sensing glucose and other nutrients, releasing enteroendocrine hormones, and transmitting signals to the brain and other target tissues, the duodenal submucosa helps coordinate insulin and glucagon secretion, nutrient uptake and utilization, and other processes involved in maintaining stable blood glucose levels.

The submucosal tissue of the duodenum primarily contains blood vessels, nerves, and connective tissue, which provide structural and supportive functions. The submucosal nerves in the duodenum are part of the enteric nervous system and have an important role in the regulation of glucose homeostasis. Submucosal nerves contain sensory neurons that can detect changes in the luminal environment of the duodenum, including the presence of nutrients such as glucose and transmit signals to the central nervous system (CNS) and enteric nervous system (ENS) and with the autonomic nervous system (ANS). This allows for coordinated neural signaling that influences glucose metabolism, including insulin secretion, hepatic glucose production, and peripheral glucose uptake. Submucosal nerves also modulate the release of hormones involved in glucose homeostasis. For example, they can stimulate the secretion of glucagon-like peptide-1 (GLP-1) and glucose-dependent insulinotropic peptide (GIP) from the enteroendocrine cells in the duodenal mucosa. These hormones play a crucial role in insulin secretion and glucose regulation. The vagus nerve, a major component of the autonomic nervous system, innervates the submucosal layer of the duodenum and can influence glucose homeostasis by regulating insulin secretion, hepatic glucose metabolism, and gut motility. It helps maintain a balance in glucose levels through the interaction between the central and enteric nervous systems. The submucosal nerves participate in the bidirectional communication between the gut and the brain, known as the gut-brain axis. This communication pathway allows for the integration of signals from the gastrointestinal tract, including glucose-related information, with the CNS. The submucosal nerves play a role in transmitting these signals and influencing central control of glucose homeostasis. It is clear that the submucosal nerves, along with other components of the enteric nervous system, play a significant role in the neural regulation of glucose metabolism.

Several devices and methods have been developed to bypass or remove a portion of the duodenum in attempts to treat diabetes. A non-surgical duodenal-jejunal bypass liner (DJBL) has been developed to mimic Roux-en-Y Gastric Bypass (RYGB)-related proximal small intestinal exclusion. The DJBL is a 60-cm-long impermeable liner which is delivered and retrieved endoscopically. Similar to RYGB, the DJBL causes significant weight loss and improvement of glycemic control. Unlike RYGB, the anatomy of the stomach and small intestine is not affected by DJBL treatment, enabling mechanistic studies focusing exclusively on the role of the proximal intestine in T2DM. FIG. 1 schematically illustrates position of a DJBL 102 positioned within a duodenum 104. The figure also shows the relative positions of a stomach 106 and an esophagus 108.

Serious adverse events related to DJBL use have been reported, such as, gastrointestinal bleeding, device migration, obstruction, and the development of liver abscesses. Liver abscesses pose the most serious complication associated with the DJBS. The Food and Drug Administration (FDA) halted the US-FDA trial due to the development of 7 liver abscesses (3.5%), which was much higher than anticipated. The cause for these liver abscesses is unclear, but the theory is that the DJBS anchors in the duodenum creates a nidus of infection, which may spread to the liver bed.

A procedure known as duodenal mucosal resurfacing (DMR) is performed using specially designed catheters which are advanced over a guidewire next to the endoscope. FIG. 2 illustrates positioning of a DMR catheter 202 positioned within a duodenum 204 during a DMR procedure. DMR is a single, minimally invasive endoscopic procedure that involves circumferential hydrothermal ablation of the duodenal mucosa resulting in subsequent regeneration of the mucosa. However, thermal ablation of duodenal mucosa, which is very thin, may result in strictures, hemorrhage, and perforation. Submucosal injection of saline is performed to prevent deep thermal injury. Additional treatment modalities known in the art, including laser, cryoablation, pulse field ablation or electroporation are also known to have been used for this procedure.

In one procedure, as shown in FIG. 3, self-assembling magnets 302 are used for suture-less compression anastomosis of the small bowel 304 that is less invasive, easily delivered, and leaves no permanent foreign body, which does occur with the conventional anastomosis technology. This technology uses a pair of magnets to create a digestive anastomosis to be performed using endoscopy without needing surgery. The device is designed to allow a portion of ingested food to move from the beginning of the small bowel jejunum to its end of the small bowel ileum, creating an enteral diversion and improving glycemic control in patients with diabetes. However, the magnets 302 could bind in undesirable portions of the small bowel and the proximal foregut might not be adequately bypassed.

The methods listed above mainly target the intestinal mucosa and do not significantly affect the glucose homeostasis mechanisms that are related to the intestinal submucosa and hence have suboptimal clinical outcomes.

Bariatric surgery has long been recognized as a potential treatment for both morbid obesity and the metabolic processes that accompany it, specifically T2D. The most performed bariatric surgeries in the United States include laparoscopic and robotic Roux-en-Y Gastric Bypass (RYGB) or Sleeve Gastrectomy (SG). While surgical treatment is based on the principles of restriction and intestinal malabsorption, evidence suggests that there are more complex mechanisms at play. Bariatric surgery has consistently been shown to improve blood glucose while allowing decreased oral hypoglycemic medications and insulin use dramatically and rapidly, effectively reversing diabetes in up to 80% of patients. In addition to early post-operative improvement in blood glucose and insulin sensitivity, bariatric surgery has also been shown to cause alterations in GI hormone release, including ghrelin, leptin, cholecystokinin (CCK), peptide-tyrosine-tyrosine (PYY), and glucagon-like peptide 1 (GLP-1), that may impact feeding behavior via the gut-brain axis, in addition to modulating euglycemia. Increases in insulin-like growth factor 1 levels (IGF-1) and decreases of plasma leptin levels have been seen in diabetic and non-diabetic morbidly obese patients. Furthermore, microbial changes in the human gut have been linked to obesity, and surgical alterations to gastrointestinal anatomy have been associated with dramatic changes in gut microbiota populations, with reversion from an “obesogenic” to a lean bacterial population.

It is speculated that T2D might be the result of an imbalance in the equilibrium between anti-incretin factors and incretins, which eventually leads to delayed insulin response and impaired insulin action. FIG. 4A schematically illustrates the imbalance in the equilibrium between anti-incretin factors 401 and incretins 403. FIG. 4B shows that the anti-incretin factors are most likely overproduced by cells 405 that produce an unknown factor with an anti-incretin effect in the proximal foregut (proximal duodenum) 402 of diabetics. Cells 407 that produce incretins are also present in the proximal foregut 402 and extend further into the small bowel 406. FIG. 4C illustrates a stomach 400 and small bowel 410 after gastric bypass surgery, depicting the duodenum 412 detached proximally from the stomach 400 and having a blind proximal pouch, and attached distally to the jejunum 414. The proximal portion of the jejunum 414 is attached to the distal end of the stomach 400. There is an increase in production of GLP-1 and other hormones from cells 407 that produce incretins, resulting in improved insulin response and action. A decrease in production of anti-incretin factor from cells 405 that produce the unknown factor with anti-incretin effect, together with the increase in incretin production, results in normalization of plasma insulin and glucose. Gastric bypass surgery is also not without its drawbacks. The procedure is invasive and complications can arise from the surgery. Further, it is not feasible to perform gastric bypass surgery in all patients suffering from diabetes.

Bariatric surgery, specifically procedures like Roux-en-Y gastric bypass (RYGB) and sleeve gastrectomy, has been shown to have a profound impact on the improvement and even remission of type 2 diabetes. Some of the mechanisms that may explain how bariatric surgery improves diabetes:

Weight Loss and Caloric Restriction: Bariatric surgery leads to significant weight loss and caloric restriction, which can directly improve insulin sensitivity and glucose control. Reduction in adipose tissue, particularly visceral fat, is associated with improved insulin signaling and glucose metabolism.

Gut Hormone Changes: Bariatric surgery alters the gut hormone milieu, which can have significant effects on glucose homeostasis. Procedures like RYGB and sleeve gastrectomy result in the rearrangement of the gastrointestinal tract, leading to changes in the production and secretion of various gut hormones. These hormonal changes include increased secretion of glucagon-like peptide-1 (GLP-1), peptide YY (PYY), and oxyntomodulin, which promote insulin secretion, enhance insulin sensitivity, suppress appetite, and regulate glucose metabolism.

Intestinal Nutrient Sensing: Bariatric surgery alters the nutrient flow and absorption patterns in the gastrointestinal tract. The rearrangement of the digestive system can result in accelerated nutrient delivery to the distal intestine, particularly the jejunum and ileum. This nutrient exposure in the lower intestine triggers specific nutrient-sensing mechanisms, leading to enhanced insulin secretion, improved glucose uptake, and regulation of hepatic glucose production.

Intestinal Microbiota Changes: Bariatric surgery has been shown to induce alterations in the gut microbiota composition, favoring a microbial profile associated with improved metabolic health. Changes in the gut microbiota after surgery can influence energy extraction from the diet, modulation of gut hormones, and inflammation, all of which impact glucose homeostasis.

Insulin-Independent Glucose-Lowering Effects: Bariatric surgery has been shown to have direct effects on glucose metabolism independent of insulin. This can include increased glucose uptake by peripheral tissues, enhanced hepatic glucose utilization, and improved insulin-independent pathways of glucose disposal.

The overall effects of bariatric surgery on diabetes are likely multifactorial and involve a combination of weight loss, hormonal changes, altered gut microbiota, and metabolic adaptations and most of these mechanisms are controlled by intestinal mucosa and submucosa.

Surgical procedures by resecting the complete intestine, target both the mucosal and submucosal mechanisms and have shown superior outcomes in improving type-2 diabetes, glucose intolerance, and related conditions. These procedures provide long-lasting and significant effects. However, it's important to note that these surgeries are highly invasive and expensive, and they carry a considerable risk of complications and mortality. Additionally, once the procedure is performed, it cannot be repeated if it fails to produce the desired outcomes. This lack of opportunity for repeat interventions is due to the accommodation or adaptation mechanisms that can occur in patients, is another significant negative with traditional surgeries.

Therefore, less invasive and more practical systems and methods that address the imbalance between anti-incretin factors and incretins by targeting both the intestinal mucosa and submucosa equally and not affecting the muscularis and serosa, and therefore treat diabetes, metabolic syndrome and various conditions associated with insulin resistance, are needed. Such systems and methods would be deployed less invasively, for example, using an endoscope, target the mucosa and submucosa and would not require the resection of a complete or full thickness organ or damage the full thickness of the intestine. Further, any foreign bodies introduced into the body during the procedure would be configured to exit the body naturally after the resection is completed, lowering the risk for complications, or requiring repeat procedures. Additionally, the procedure could be performed on an outpatient basis, lowering costs and complexity. Minimizing or eliminating use of thermal or other ablative energy required for mucosal ablation procedures will minimize or eliminate complications such as strictures, bleeding, perforation and pancreatitis.

### SUMMARY

The following embodiments and aspects thereof are described and illustrated in conjunction with systems, tools and methods, which are meant to be exemplary and illustrative, and not limiting in scope. The present application discloses numerous embodiments.

The present specification discloses a device for resecting a body tissue, comprising: a cap configured to attach to a distal end of an endoscope wherein the cap comprises a central lumen; a suction source configured to apply a negative pressure through the central lumen of the cap; and at least one resection component positioned around an outer surface of the cap, wherein the at least one resection component has an inner surface comprising at least one anchor, wherein the at least one resection component comprises a first material, the at least one anchor comprises a second material, and wherein the first material is different from the second material, wherein the at least one resection component is configured to change from a first configuration when positioned on the cap to a second configuration once deployed, wherein, in the second configuration, the at least one resection component is configured to encircle a portion of said body tissue with the at least one anchor contacting the tissue to secure the resection component to constrict the tissue and reduce blood flow to the tissue.

Optionally, the at least one resection component comprises a band, a loop, an O-ring, an elastic circle, or any other component adapted to have a first configuration with a circumference of M when subjected to a pressure and adapted to automatically transition to a second configuration with a circumference of N when the pressure is removed, where N is less than M.

Optionally, the anchor comprises a wire of the shape-memory material having at least one first portion extending into the at least one resection component and at least one second portion extending out of and toward a center of the at least one resection component. Optionally, the shape memory material comprises Nitinol. Optionally, in the second configuration, the at least one second portion of the wire is configured to anchor into the body tissue. Optionally, in the second configuration, the at least one second portion of the wire is in the shape of a spike and is configured to pierce the body tissue.

Optionally, the device comprises 1 to 10 resection components.

Optionally, the device further comprises a catheter extending between the cap and the suction source.

The present specification also discloses a method for resecting a body tissue in a patient, comprising: providing a device configured to pass through an endoscope comprising: a cap positioned on the distal end of the device, wherein the cap comprises a central lumen; and at least one resection component positioned around an outer surface of the cap, wherein the at least one resection component has an inner surface comprising at least one anchor, wherein the at least one resection component is configured to change from a first configuration when positioned on the cap to a second configuration once deployed off the cap, wherein, in the second configuration, the at least one resection component is configured to encircle a portion of said body tissue, anchor into the tissue using the anchor, and to constrict the tissue and reduce blood flow to the tissue; inserting the device through the endoscope into the patient and advancing the device such that a distal end of the device is positioned in a gastrointestinal tract of the patient; activating a suction source to apply a negative pressure through the central lumen of the cap; drawing a portion of body tissue into the central lumen of the cap, wherein the portion includes at least 1 square centimeters of body tissue; deploying at least one resection component from the device around a base of the body tissue; removing the device from the patient; permitting the at least one resection component to remain around the body tissue and to cut off blood flow to the body tissue.

Optionally, the at least one resection component comprises a band, a loop, an O-ring, an elastic circle, or any other component adapted to have a first configuration with a circumference of M when subjected to a pressure and adapted to automatically transition to a second configuration with a circumference of N when the pressure is removed, where N is less than M.

Optionally, the anchor comprises a wire of a shape-memory material having at least one first portion extending into the at least one resection component and at least one second portion extending out of and toward a center of the at least one resection component. Optionally, the shape memory material comprises Nitinol. Optionally, in the second configuration, the at least one second portion of the wire is configured to anchor into the body tissue. Optionally, in the second configuration, the at least one second portion of the wire is in the shape of a spike and is configured to pierce the body tissue.

Optionally, the device comprises 1 to 10 resection components.

Optionally, the device further comprises a catheter extending between the cap and the suction source.

Optionally, the method is used to treat at least one of excess weight, obesity, an eating disorder, metabolic syndrome, dyslipidemia, diabetes, polycystic ovarian disease, fatty liver disease, non-alcoholic fatty liver disease, or non-alcoholic steatohepatitis disease.

Optionally, the method further comprises determining a therapeutic endpoint after the resection.

Optionally, the therapeutic endpoint is at least one of a total body weight of the patient decreases by at least 1% relative to a total body weight of the patient before resection, an excess body weight of the patient decreases by at least 1% relative to an excess body weight of the patient before resection, a total body weight of the patient decreases by at least 1% relative to a total body weight of the patient before resection and a well-being level of the patient does not decrease more than 5% relative to a well-being level of the patient before resection, an excess body weight of the patient decreases by at least 1% relative to an excess body weight of the patient before resection and a well-being level of the patient does not decrease more than 5% relative to a well-being level of the patient before resection, a pre-prandial ghrelin level of the patient decreases by at least 1% relative to a pre-prandial ghrelin level of the patient before resection, a post-prandial ghrelin level of the patient decreases by at least 1% relative to a post-prandial ghrelin level of the patient before resection, or an exercise output of the patient increases by at least 1% relative to an exercise output of the patient before resection.

Optionally, the therapeutic endpoint is at least one of a glucagon-like peptide-1 level of the patient increases by at least 1% relative to a glucagon-like peptide-1 level of the patient before resection, a leptin level of the patient increases by at least 1% relative to a leptin level of the patient before resection, the patient's appetite decreases, over a predefined period of time, relative to the patient's appetite before resection, a peptide YY level of the patient increases by at least 1% relative to a peptide YY level of the patient before resection, a lipopolysaccharide level of the patient decreases by at least 1% relative to a lipopolysaccharide level of the patient before resection, a motilin-related peptide level of the patient decreases by at least 1% relative to a motilin-related peptide level of the patient before resection, a cholecystokinin level of the patient increases by at least 1% relative to a cholecystokinin level of the patient before resection, a resting metabolic rate of the patient increases by at least 1% relative to a resting metabolic rate of the patient before resection, a plasma-beta endorphin level of the patient increases by at least 1% relative to a plasma-beta endorphin level of the patient before resection, an HbA1c level of the patient decreases by at least 0.3% relative to an HbA1c level of the patient before resection, a triglyceride level of the patient decreases by at least 1% relative to a triglyceride level of the patient before resection, a total blood cholesterol level of the patient decreases by at least 1% relative to a total blood cholesterol level of the patient before resection, or a glycemia level of the patient decreases by at least 1% relative to a glycemia level of the patient before resection.

Optionally, the therapeutic endpoint is a cumulative daily dose of the patient's antidiabetic medications decreases by at least 10% relative to a cumulative daily dose of the patient's antidiabetic medications before resection or 10% of the patients eliminate the use of one or more of their antidiabetic medications being used before resection.

Optionally, the therapeutic endpoint is at least one of a 10% decrease in either ALT or AST levels relative to ALT or AST levels before resection, at least a 10% improvement in serum ferritin level or an absolute serum ferritin level of less than 1.5 ULN (upper limit normal) relative to serum ferritin levels before resection, at least a 5% improvement in hepatic steatosis (HS) or less than 5% HS relative to HS levels before resection, as measured on liver biopsy, at least a 5% improvement in HS or less than 5% HS relative to HS levels before resection, as measured by magnetic resonance (MR) imaging, either by spectroscopy or proton density fat fraction, at least a 5% improvement in an NAFLD Fibrosis Score (NFS) relative to an NFS before resection, at least a 5% improvement in an NAFLD Activity Score (NAS) relative to an NAS before resection, at least a 5% improvement in a Steatosis Activity Fibrosis (SAF) score relative to an SAF score before resection, or at least a 5% decrease in a mean annual fibrosis progression rate relative to a mean annual fibrosis progression rate before resection, as measured by histology.

The present specification also discloses a device for resecting a body tissue which include the gastric intestinal mucosa and submucosa without significantly affecting the muscularis or serosa, comprising: a cap configured to pass through a distal end of an endoscope wherein the cap comprises a central lumen; a suction source configured to apply a negative pressure through the central lumen of the cap; and at least one elastic ligation device such as a rubber band positioned around an outer surface of the cap, wherein the at least one band has an inner surface comprising at least one anchor, wherein the at least one band is configured to change from a first configuration when positioned on the cap to a second configuration once deployed, wherein, in the second configuration, the at least one band is configured to encircle a portion of said body tissue with the at least one anchor contacting the tissue to secure the band to constrict the tissue and reduce blood flow to the tissue. The device resects and/or affects the mucosa and submucosa in an equivalent amount to achieve the desired effect. Stated differently, the device resects and/or affects the functionality of the mucosa and the functionality of the submucosa in an equivalent amount, such as a decrease in functionality, including metabolic function, in a range of 10% to 90% and any increment therein, to achieve the desired effect.

Optionally, the anchor comprises a wire of the shape-memory material having at least one first portion extending into the at least one band and at least one second portion extending out of and toward a center of the at least one band. Optionally, the shape memory material comprises Nitinol. Optionally, in the second configuration, the at least one second portion of the wire is configured to anchor into the body tissue. Optionally, in the second configuration, the at least one second portion of the wire is in the shape of a spike and is configured to pierce the body tissue.

Optionally, the device comprises 1 to 100 bands or ligation devices.

Optionally, the device further comprises a wire or a catheter extending between the cap and the suction source to help deploy the bands.

The present specification also discloses a method for resecting a body tissue in a patient, comprising: providing a device configured to pass through an endoscope comprising: a cap positioned on the distal end of the device, wherein the cap comprises a central lumen; and at least one band or ligation device positioned around an outer surface of the cap, wherein the at least one band has an inner surface comprising at least one anchor, wherein the at least one band or ligation device is configured to change from a first configuration when positioned on the cap to a second configuration once deployed off the cap, wherein, in the second configuration, the at least one band is configured to encircle a portion of said body tissue, anchor into the tissue using the anchor, and to constrict the tissue and reduce blood flow to the tissue; inserting the device through the endoscope into the patient and advancing the device such that a distal end of the device is positioned in a duodenum of the patient; activating a suction source to apply a negative pressure through the central lumen of the cap; drawing a portion of body tissue into the central lumen of the cap, wherein the portion includes at least 5 square centimeters of body tissue; deploying at least one band from the device around a base of the body tissue; removing the device from the patient; permitting the at least one band to remain around the body tissue and to cut off blood flow to the body tissue. The portion of the body tissue constitute of at least a mucosal tissue and a submucosal tissue.

Optionally, the anchor comprises a wire of a shape-memory material having at least one first portion extending into the at least one band or ligation device and at least one second portion extending out of and toward a center of the at least one band or a ligation device. Optionally, the shape memory material comprises Nitinol. Optionally, in the second configuration, the at least one second portion of the wire is configured to anchor into the body tissue. Optionally, in the second configuration, the at least one second portion of the wire is in the shape of a spike and is configured to pierce the body tissue. Optionally, the devise comprises 1 to 100 bands. Optionally, the device further comprises a catheter extending between the cap and the suction source.

The method may be used to treat at least one of excess weight, obesity, an eating disorder, metabolic syndrome, dyslipidemia, diabetes, polycystic ovarian disease, fatty liver disease, non-alcoholic fatty liver disease, or non-alcoholic steatohepatitis disease or any other disease associated with insulin resistance.

Optionally, the method further comprises determining a therapeutic endpoint after the resection. The therapeutic endpoint may be at least one of a total body weight of the patient decreases by at least 1% relative to a total body weight of the patient before resection, an excess body weight of the patient decreases by at least 1% relative to an excess body weight of the patient before resection, a total body weight of the patient decreases by at least 1% relative to a total body weight of the patient before resection and a well-being level of the patient does not decrease more than 5% relative to a well-being level of the patient before resection, an excess body weight of the patient decreases by at least 1% relative to an excess body weight of the patient before resection and a well-being level of the patient does not decrease more than 5% relative to a well-being level of the patient before resection, a pre-prandial ghrelin level of the patient decreases by at least 1% relative to a pre-prandial ghrelin level of the patient before resection, a post-prandial ghrelin level of the patient decreases by at least 1% relative to a post-prandial ghrelin level of the patient before resection, or an exercise output of the patient increases by at least 1% relative to an exercise output of the patient before resection.

The therapeutic endpoint may also be at least one of a glucagon-like peptide-1 level of the patient increases by at least 1% relative to a glucagon-like peptide-1 level of the patient before resection, a leptin level of the patient increases by at least 1% relative to a leptin level of the patient before resection, the patient's appetite decreases, over a predefined period of time, relative to the patient's appetite before resection, a peptide YY level of the patient increases by at least 1% relative to a peptide YY level of the patient before resection, a lipopolysaccharide level of the patient decreases by at least 1% relative to a lipopolysaccharide level of the patient before resection, a motilin-related peptide level of the patient decreases by at least 1% relative to a motilin-related peptide level of the patient before resection, a cholecystokinin level of the patient increases by at least 1% relative to a cholecystokinin level of the patient before resection, a resting metabolic rate of the patient increases by at least 1% relative to a resting metabolic rate of the patient before resection, a plasma-beta endorphin level of the patient increases by at least 1% relative to a plasma-beta endorphin level of the patient before resection, an HbA1c level of the patient decreases by at least 0.3% relative to an HbA1c level of the patient before resection, a triglyceride level of the patient decreases by at least 1% relative to a triglyceride level of the patient before resection, a total blood cholesterol level of the patient decreases by at least 1% relative to a total blood cholesterol level of the patient before resection, or a glycemia level of the patient decreases by at least 1% relative to a glycemia level of the patient before resection.

The therapeutic endpoint may also be at least one of a composition of the person's gut microbiota modulates from a first state before resection to a second state after resection, wherein the first state has a first level of bacteroidetes and a first level of firmicutes, wherein the second state has a second level of bacteroidetes and a second level of firmicutes, wherein the second level of bacteroidetes is greater than the first level of bacteroidetes by at least 3%, and wherein the second level of firmicutes is less than the first level of firmicutes by at least 3%, or a cumulative daily dose of the patient's antidiabetic medications decreases by at least 10% relative to a cumulative daily dose of the patient's antidiabetic medications before resection.

The therapeutic endpoint may also be at least one of a 10% decrease in either ALT or AST levels relative to ALT or AST levels before resection, at least a 10% improvement in serum ferritin level or an absolute serum ferritin level of less than 1.5 ULN (upper limit normal) relative to serum ferritin levels before resection, at least a 5% improvement in hepatic steatosis (HS) or less than 5% HS relative to HS levels before resection, as measured on liver biopsy, at least a 5% improvement in HS or less than 5% HS relative to HS levels before resection, as measured by magnetic resonance (MR) imaging, either by spectroscopy or proton density fat fraction, at least a 5% improvement in an NAFLD Fibrosis Score (NFS) relative to an NFS before resection, at least a 5% improvement in an NAFLD Activity Score (NAS) relative to an NAS before resection, at least a 5% improvement in a Steatosis Activity Fibrosis (SAF) score relative to an SAF score before resection, or at least a 5% decrease in a mean annual fibrosis progression rate relative to a mean annual fibrosis progression rate before resection, as measured by histology.

The therapeutic endpoint may also be at least one of a 20% decrease in the dose of oral antidiabetic drugs (OAD) or a reduction in the dose of OAD in at least 20% of the patients.

The therapeutic endpoint may also be at least one of a 20% decrease in the dose of insulin or a reduction in the dose of insulin in at least 20% of the patients or preventing at least 10% of patients from progressing from OAD to insulin therapy.

The therapeutic endpoint may also be at least one of decreasing the incidence of severe hypoglycemic events by 5% or decreasing the incidence of severe hyperglycemic events in at least 25% of the patients.

The aforementioned and other embodiments of the present specification shall be described in greater depth in the drawings and detailed description provided below.

### BRIEF DESCRIPTION OF THE DRAWINGS

The accompanying drawings illustrate various embodiments of systems, methods, and embodiments of various other aspects of the disclosure. Any person with ordinary skills in the art will appreciate that the illustrated element boundaries (e.g. boxes, groups of boxes, or other shapes) in the figures represent one example of the boundaries. It may be that in some examples one element may be designed as multiple elements or that multiple elements may be designed as one element. In some examples, an element shown as an internal component of one element may be implemented as an external component in another and vice versa. Furthermore, elements may not be drawn to scale. Non-limiting and non-exhaustive descriptions are described with reference to the following drawings. The components in the figures are not necessarily to scale, emphasis instead being placed upon illustrating principles.

FIG. 1 schematically illustrates a duodenal-jejunal bypass liner (DJBL) positioned within a duodenum;

FIG. 2 illustrates a catheter positioned within a duodenum during a duodenal mucosal resurfacing (DMR) procedure;

FIG. 3 illustrates a system that uses self-assembling magnets for suture-less compression anastomosis positioned within a small bowel;

FIG. 4A illustrates an imbalance in the equilibrium between anti-incretin factors and incretins believed to have a causative role in the development of diabetes;

FIG. 4B illustrates a foregut depicting the location of anti-incretin factor producing cells and incretin producing cells in diabetic patients;

FIG. 4C illustrates a stomach and small bowel after gastric bypass surgery;

FIG. 5A illustrates an anatomical layout of the proximal digestive system;

FIG. 5B illustrates a resection device in accordance with some embodiments of the present specification;

FIG. 6A illustrates a distal end of an endoscopic device comprising resection components or bands configured for resecting portions of foregut mucosa and submucosa, in accordance with some embodiments of the present specification;

FIG. 6B illustrates a portion of foregut mucosa with a resection component or band around a base of the mucosal and submucosal portion after having been deployed by the device of FIG. 6A, in accordance with some embodiments of the present specification;

FIG. 6C illustrates a schematic cross-sectional view of a portion of a proximal foregut with the distal end of an endoscopic device positioned within a duodenum and a plurality of foregut mucosa and submucosal portions with resection components or bands positioned around bases of the mucosal and submucosal portions, in accordance with some embodiments of the present specification;

FIG. 6D is a flowchart listing the steps involved in using an endoscopic device to resect mucosal and submucosal tissue, in accordance with one embodiment of the present specification;

FIG. 7A illustrates a distal end of an endoscopic device for cap assisted mucosal and submucosal resection, in accordance with some embodiments of the present specification;

FIG. 7B illustrates the device of FIG. 7A with a portion of foregut mucosa and submucosa pulled into a cap of the device;

FIG. 7C illustrates the device of FIGS. 7A and 7B with the portion of foregut mucosa and submucosa released from the cap and a snare loop positioned around a base of the mucosal and submucosal portion;

FIG. 7D is a flowchart listing the steps involved in using an endoscopic device to resect tissue, in accordance with another embodiment of the present specification;

FIG. 8A illustrates the distal end of an endoscopic device with a resection component or band positioned around a base of a portion of foregut mucosa and submucosa, in accordance with some embodiments of the present specification;

FIG. 8B illustrates the device of FIG. 8A with a snare loop positioned around the base of the portion of foregut mucosa and submucosa, in accordance with some embodiments of the present specification;

FIG. 8C illustrates the device of FIGS. 8A and 8B, depicting withdrawal of the snare loop along with the resected portion of foregut mucosa and submucosa, in accordance with some embodiments of the present specification;

FIG. 8D is a flowchart listing the steps involved in using an endoscopic device to resect tissue, in accordance with another embodiment of the present specification;

FIG. 9A illustrates an endoscopic device configured to use negative pressure to lift a portion of foregut mucosa and submucosa and deploy a ligation/resection component or band, in accordance with some embodiments of the present specification;

FIG. 9B is a flowchart listing the steps involved in using an endoscopic device to resect tissue, in accordance with another embodiment of the present specification;

FIG. 10A illustrates a distal end of an endoscopic device having a plurality of resection components/ligation bands with shape-memory spikes positioned on the distal end, in accordance with some embodiments of the present specification;

FIG. 10B illustrates a cross-sectional view of a resection component or band of the device of FIG. 10A;

FIG. 10C illustrates a front perspective view of multiple resection components or bands showing spikes of a shape-memory wire that extends out from a body of each resection component or band toward a center of each resection component or band, in accordance with some embodiments of the present specification;

FIG. 11A illustrates a resection component or band with a shape-memory wire positioned inside the resection component or band in expanded and contracted configurations, in accordance with some embodiments of the present specification;

FIG. 11B illustrates a resection component or band with a shape-memory wire positioned inside the resection component or band in expanded and contracted configurations, in accordance with other embodiments of the present specification;

FIG. 11C illustrates a resection component or band with a shape-memory wire positioned inside the resection component or band in a contracted configuration, in accordance with still other embodiments of the present specification;

FIG. 12A illustrates a resection component or band with a plurality of shape-memory wire portions positioned along an inner circumference of the resection component or band in expanded and contracted configurations, in accordance with some embodiments of the present specification;

FIG. 12B illustrates a resection component or band with a plurality of shape-memory wire or stainless steel extensions positioned along an inner circumference of the resection component or band in expanded and contracted configurations, in accordance with some embodiments of the present specification;

FIG. 13A illustrates a resection component or band with a plurality of protrusions extending toward a center of the resection component or band in expanded and contracted configurations, in accordance with some embodiments of the present specification;

FIG. 13B illustrates a resection component or band with a plurality of protrusions extending toward a center of the resection component or band in expanded and contracted configurations, in accordance with other embodiments of the present specification;

FIG. 14A illustrates a distal end of an endoscopic device comprising a cylindrical cap with at least two resection components or elastic bands positioned around an external surface of cap, in accordance with some embodiments of the present specification;

FIG. 14B illustrates resection components or bands of device FIG. 14A deployed around bases of foregut mucosal and submucosal portions, in accordance with some embodiments of the present specification;

FIG. 15A illustrates an endoscopic resection device positioned to cut a resected tissue from a ligation site, while simultaneously pulling the resected tissue from its proximal side, in accordance with some embodiments of the present specification;

FIG. 15B illustrates twisting and pulling of the resected tissue using the endoscopic resection device of FIG. 15A, in accordance with some embodiments of the present specification;

FIG. 15C illustrates loss of contact of resected tissue from an intestinal tissue surface using the endoscopic resection device of FIG. 15A;

FIG. 15D illustrates multiple views of a rotating cutting blade deployed at the site of ligation to assist in separation of resected tissue from intestinal tissue surface, in accordance with an embodiment of the present specification; and

FIG. 15E is a flow chart illustrating an exemplary set of steps used to achieve morcellation using the embodiments of FIGS. 15A to 15D, in accordance with some embodiments of the present specification.

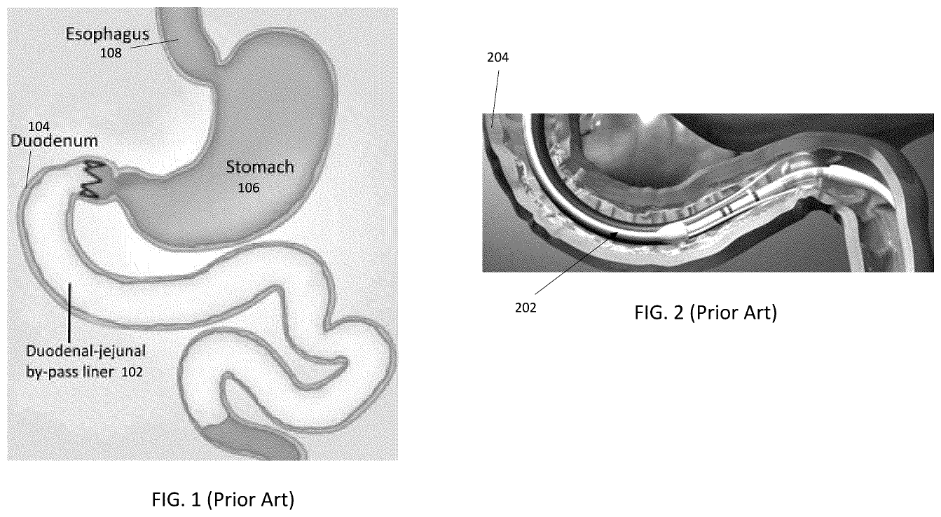

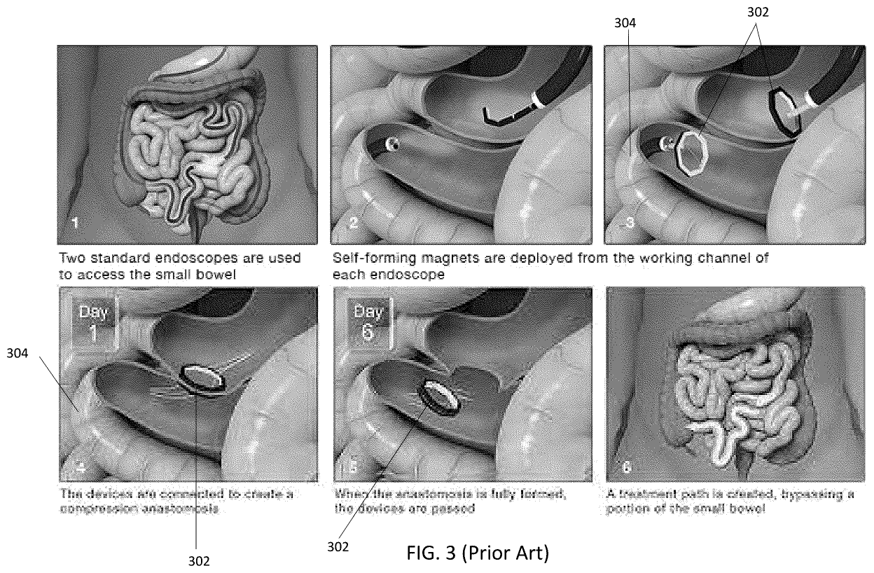

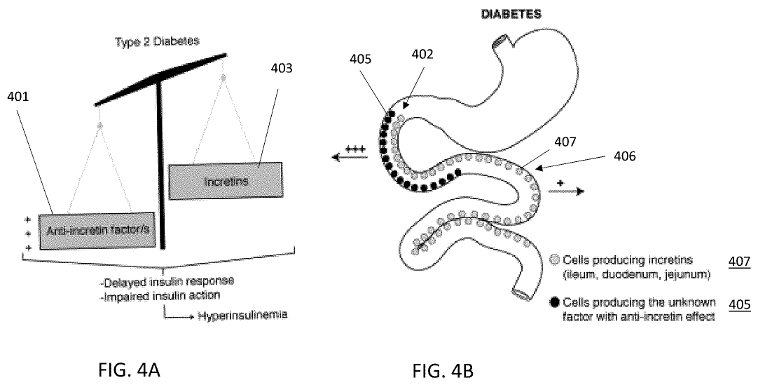

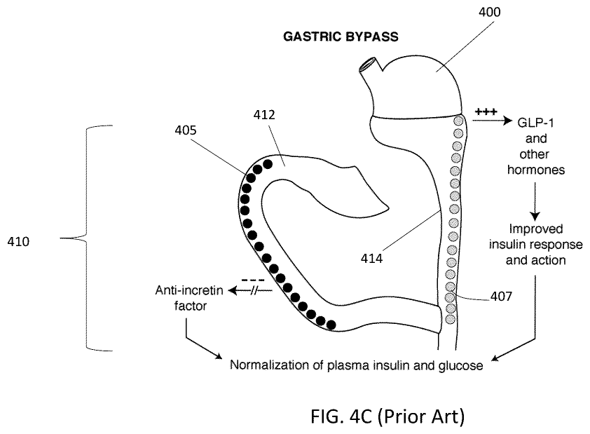

### DETAILED DESCRIPTION

The present specification is directed towards methods and systems for proximal foregut mucosal and submucosal tissue removal or resection, and more specifically, duodenal mucosal and submucosal removal or resection. In embodiments, the imbalance in the equilibrium between anti-incretin factors and incretins because of overproduction of anti-incretins is addressed by the tissue removal or resection systems and methods of the present specification. Specifically, cells in the duodenum responsible to produce factors with anti-incretin effects are removed by the selective resection of mucosal and submucosal tissue in the duodenum without removing or resecting the muscularis or serosal tissue as done during a surgical procedure. The resection or tissue removal procedure is performed in the proximal foregut of diabetic patients. Resection is a medical procedure that is used to remove anatomical structures from the body. In embodiments of the present specification, portions of a mucosal and submucosal tissue of proximal foregut, or duodenum, are resected or removed. In some embodiments, mucosal and sub-mucosal tissue may be resected.

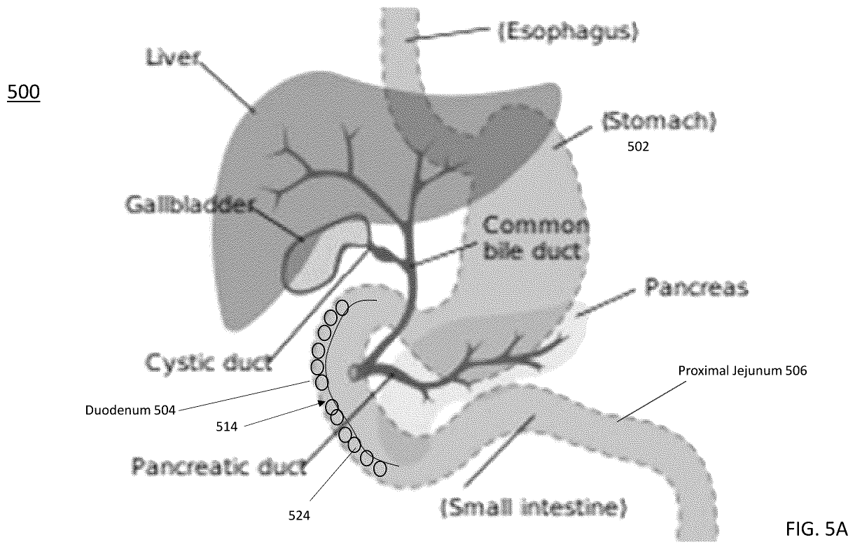

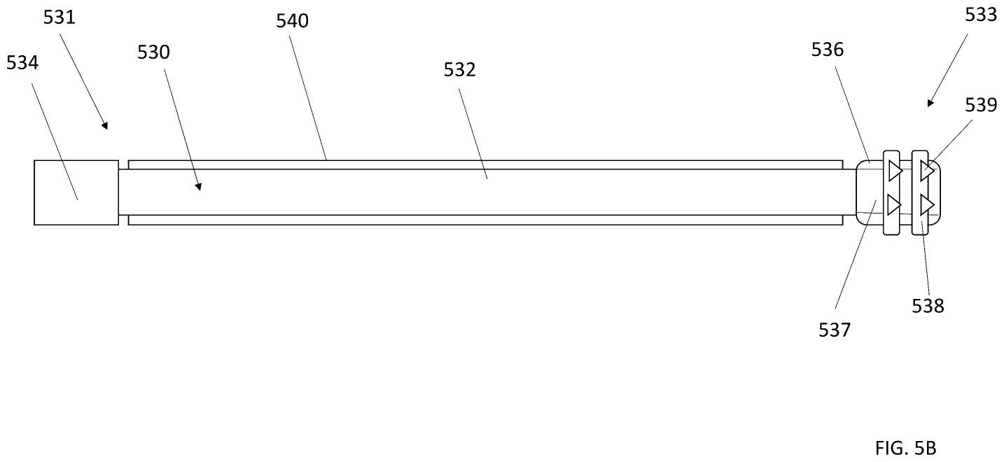

FIG. 5A illustrates an anatomical layout of a digestive system 500. The proximal foregut in the digestive system 500 includes portions of the stomach 502, duodenum 504, and proximal jejunum 506. Resecting the layer 514 of mucous glands that line the inner wall of the proximal foregut eliminate the cells 524 that produce anti-incretin factor/s. Additionally, the stimulation of more distal intestinal mucosa results in earlier and/or increased production of GLP-1 and other hormones. Additionally, resection of the intestinal submucosa through both neural (CNS, ANS, ENS) pathways and through the neurohumoral pathways as listed above regulate glucose homeostasis. Non-thermal resection or removal of the proximal foregut mucosa and submucosa results in a much safer procedure relative to currently known procedures, reducing the side-effects of pain, bleeding, perforation or stricture formation. Targeting both the mucosal and submucosal tissues will have a more profound synergistic effect on glucose homeostasis than targeting just the mucosal layer.

In some embodiments, resection of the foregut mucosa and/or submucosa is performed using one of the techniques of endoscopic band ligation, endoscopic mucosal resection, or endoscopic submucosal dissection. Elastic or rubber band ligation involves the deploying a constricting elastic resection component, device, member, material, or band about the mucosal tissue surface that is to be resected. Deploying the band around the tissue occludes distal blood supply to the tissue so that the affected portion slowly dies, sloughs off, and is passed out from the body. Endoscopic band ligation procedures involve use of a ligation device that is affixed to the distal end of an endoscope. One or more elastic bands are positioned at the distal end for this purpose. During deployment, the most distal band is stretched outwardly beyond its relaxed configuration, and placed upon a surface of the mucosa in the proximal foregut. In some embodiments, a shape-memory material, such as Nitinol, is positioned along with or within the loop of the band. The shape-memory material contracts upon deployment, along with constricting of the elastic band to its non-stretched configuration, and assists in keeping the band positioned about the target tissue until the ischemic necrosis and/or resection is complete, preventing premature dislodging of the bands or resection devices. In embodiments, the shape-memory material is configured in different forms to provide an anchor into the mucosal surface. The anchoring and constricting of the shape-memory material and the constricting action of the elastic band enables the loop of the elastic band to gather a portion of the encircled mucosal surface and submucosal tissue into a nodule. The continued constriction action of the anchor and the band apply pressure on the gathered target tissue surface, resulting in the cutting-off of blood supply to that portion of the tissue and eventual death and sloughing off of that tissue portion. Resecting and removing both the mucosal and submucosal tissue will target glucose homeostasis mechanisms that reside both the mucosal and submucosal layer, resulting in more profound synergistic effect on glucose metabolism.

Referring to FIG. 5B, a resection device 530 in accordance with embodiments of the present specification, particularly the “endoscopic devices” defined in the present specification, comprises a catheter 532 having a proximal end 531 and a distal end 533 with a suction source 534 at the proximal end 531 of the catheter 532 and a cap 536 at the distal end 533 of the catheter 532. The catheter 532 is configured to pass through a working channel of an endoscope 540. The cap 536 is configured to be passed through a distal end of the endoscope 540 and comprises a central lumen 537. The suction source 534 is activated to apply a negative pressure through the central lumen 537 of the cap 536 to draw tissue into the cap. At least one resection component or band 538 is positioned around an outer surface of the cap 536 and is configured to change from a first configuration when positioned on the cap 536 to a second configuration once deployed. In the second configuration, the at least one band 538 is configured to encircle a portion of body tissue to constrict the tissue and reduce blood flow to the tissue. The tissue eventually dies, sloughs off, and is passed by the gastrointestinal tract. In some embodiments, the at least one band 538 includes at least one anchoring mechanism 539 to assist in keeping the at least one band 538 secured to the tissue. In some embodiments, the at least one anchor 539 comprises a shape-memory wire or coil. In some embodiments, the shape-memory material is Nitinol. The resection of the foregut mucosa and/or submucosa triggers regeneration of healthy foregut mucosa and/or submucosa which will not have the diseased cells resulting in insulin resistance and dysregulated glucose homeostasis.

Foregut mucosal or submucosal resection and regeneration of normal/healthy foregut mucosa and or submucosa results in and increases the enteroglucagon and gastroinhibitory peptide response to oral glucose, increases insulin-like growth factor 1 levels (IGF-1), and lowers plasma leptin levels in diabetic and nondiabetic morbidly obese patients, similar to what is seen with gastric bypass surgery. Gastric bypass achieves normalization of plasma insulin and glucose by avoiding stimulation of cells producing the unknown factor with anti-incretin effect, in addition to earlier and/or increased GLP-1 production along with other hormones. Postprandial reduction of pancreatic polypeptide may also occur. Foregut mucosal/submucosal resection reduces leptin levels before weight loss occurs and increases the enteroglucagon response to a glucose test, similar to the results seen with biliopancreatic diversion. Embodiments of the present specification decrease plasma lipid levels similarly to that in biliopancreatic diversion in some patients. High levels of plasma glucagon-like peptide 1, reported after jejunoileal bypass, are also seen with foregut mucosal/submucosal resection using the devices and methods of the present specification. The high levels of plasma glucagon-like peptide 1 plays a role in the mechanism of diabetes control after bariatric surgeries and also results in improvement or resolution of many conditions associated with insulin resistance.

The present specification is directed towards multiple embodiments. The following disclosure is provided in order to enable a person having ordinary skill in the art to practice the invention. Language used in this specification should not be interpreted as a general disavowal of any one specific embodiment or used to limit the claims beyond the meaning of the terms used therein. The general principles defined herein may be applied to other embodiments and applications without departing from the spirit and scope of the invention. Also, the terminology and phraseology used is for the purpose of describing exemplary embodiments and should not be considered limiting. Thus, the present invention is to be accorded the widest scope encompassing numerous alternatives, modifications and equivalents consistent with the principles and features disclosed. For purpose of clarity, details relating to technical material that is known in the technical fields related to the invention have not been described in detail so as not to unnecessarily obscure the present invention.

In the description and claims of the application, each of the words “comprise”, “include”, “have”, “contain”, and forms thereof, are not necessarily limited to members in a list with which the words may be associated. Thus, they are intended to be equivalent in meaning and be open-ended in that an item or items following any one of these words is not meant to be an exhaustive listing of such item or items, or meant to be limited to only the listed item or items. It should be noted herein that any feature or component described in association with a specific embodiment may be used and implemented with any other embodiment unless clearly indicated otherwise.

It must also be noted that as used herein and in the appended claims, the singular forms “a,” “an,” and “the” include plural references unless the context dictates otherwise. Although any systems and methods similar or equivalent to those described herein can be used in the practice or testing of embodiments of the present disclosure, the preferred, systems and methods are now described.

In the present specification, the term “resection component” is defined as any elastic resection component, device, member, or material that is configured to be positioned about a tissue surface, for example, via deployment from a deployment device, to encircle or wrap around a portion of that tissue which is to be resected. A resection component includes a band, a loop, an O-ring, an elastic circle, or any other component adapted to have a first configuration with a circumference of M when subjected to a pressure and adapted to automatically transition to a second configuration with a circumference of N when the pressure is removed, where N is less than M.

Insulin resistance can be assessed through various clinical and laboratory parameters. Some commonly used parameters to measure insulin resistance include:

Fasting Insulin: Fasting insulin levels are measured after an overnight fast and provide an indication of basal insulin secretion. Elevated fasting insulin levels are often associated with insulin resistance.

Fasting Glucose: Fasting blood glucose levels are measured to evaluate the fasting state of glucose metabolism. Insulin resistance can lead to impaired fasting glucose levels, indicating a reduced ability of insulin to promote glucose uptake.

Homeostatic Model Assessment of Insulin Resistance (HOMA-IR): HOMA-IR is a mathematical formula that calculates insulin resistance using fasting glucose and fasting insulin levels. It provides an estimate of insulin sensitivity based on the assumption that the liver is the primary site of insulin resistance.

Oral Glucose Tolerance Test (OGTT): An OGTT involves measuring blood glucose and insulin levels before and after consuming a glucose solution. The response of blood glucose and insulin following glucose ingestion can help assess insulin resistance.

Insulin Sensitivity Index (ISI): ISI is a measure of whole-body insulin sensitivity and can be derived from either a hyperinsulinemic-euglycemic clamp technique or frequently sampled intravenous glucose tolerance test (FSIVGTT). It provides a quantitative assessment of insulin sensitivity.

Quantitative Insulin Sensitivity Check Index (QUICKI): QUICKI is a formula that uses fasting glucose and fasting insulin levels to estimate insulin sensitivity. It is based on the inverse relationship between insulin sensitivity and fasting insulin levels.

Matsuda Index: The Matsuda Index is calculated from glucose and insulin values obtained during an OGTT. It provides an estimate of insulin sensitivity by assessing the ability of insulin to facilitate glucose disposal.

Adipose Tissue Insulin Resistance (Adipo-IR): Adipo-IR is a measure of insulin resistance specific to adipose tissue. It is calculated using fasting plasma insulin and adiponectin levels, with lower adiponectin levels indicating higher adipose tissue insulin resistance.

Insulin Clamp Technique: The hyperinsulinemic-euglycemic clamp is considered the gold standard for measuring insulin sensitivity. It involves infusing insulin intravenously at a constant rate while maintaining blood glucose levels at a stable, predetermined level.

Lipid Profile: Insulin resistance is often associated with alterations in lipid metabolism. Assessing parameters such as triglycerides, high-density lipoprotein (HDL) cholesterol, and low-density lipoprotein (LDL) cholesterol can provide insights into the metabolic abnormalities associated with insulin resistance.

Waist Circumference and Body Mass Index (BMI): Central obesity is strongly linked to insulin resistance. Waist circumference and BMI are simple anthropometric measures used to assess adiposity and estimate the risk of insulin resistance.

One of more of these parameters can be used to provide a comprehensive evaluation of insulin resistance. Different techniques and indices may be employed in research and clinical settings based on availability and specific research or diagnostic purposes. An improvement in insulin resistance of 10% or more measured using any of these parameters is concerned clinically significant and meaningful.

The severity of non-alcoholic fatty liver disease (NAFLD) is assessed using several clinical and laboratory tests. These tests help in evaluating the degree of liver damage, inflammation, fibrosis, and other related aspects. Commonly used tests used to measure NAFLD severity include:

Liver Function Tests: Elevated ALT levels are a marker of liver inflammation and injury. Higher ALT levels often indicate more severe liver damage in NAFLD.

Imaging Studies—Ultrasonography is a non-invasive imaging technique that can detect fat accumulation in the liver and is initial screening tool for NAFLD. Computed Tomography (CT) Scan provides detailed images of the liver and help evaluate the extent of fat deposition and fibrosis. Magnetic Resonance Imaging (MRI) can assess liver fat content, fibrosis, and inflammation, providing more precise information about the severity of NAFLD. Transient Elastography (FibroScan) measures liver stiffness, which is an indirect marker of fibrosis severity. Higher liver stiffness values indicate more advanced fibrosis.

Biomarkers of Fibrosis and Inflammation include Fibrosis-4 Index (FIB-4) is a simple calculation that combines age, AST (aspartate aminotransferase) levels, ALT levels, and platelet count to estimate the degree of liver fibrosis. Higher FIB-4 scores are associated with more severe fibrosis. The NAFLD Fibrosis Score uses several clinical parameters, including age, BMI, diabetes status, platelet count, albumin levels, and AST/ALT ratio, to predict the presence and severity of liver fibrosis. Enhanced Liver Fibrosis (ELF) Test measures three serum markers (hyaluronic acid, procollagen III N-terminal peptide, and tissue inhibitor of metalloproteinase 1) associated with liver fibrosis. It provides a numerical score indicating fibrosis severity.

Liver Biopsy is considered the gold standard for assessing NAFLD severity. A small sample of liver tissue is obtained and examined under a microscope to determine the degree of fat accumulation, inflammation, hepatocyte injury, and fibrosis.

The selection of tests depends on the clinical context, resources, and individual patient factors. Non-invasive methods are preferred whenever possible, but in some cases, a liver biopsy may be necessary to provide a definitive assessment of NAFLD severity.

One of more of these parameters can be used to provide a comprehensive evaluation of NAFLD. Different techniques and indices may be employed in research and clinical settings based on availability and specific research or diagnostic purposes. An improvement in insulin resistance of 10% or more measured using any of these parameters is concerned clinically significant and meaningful.

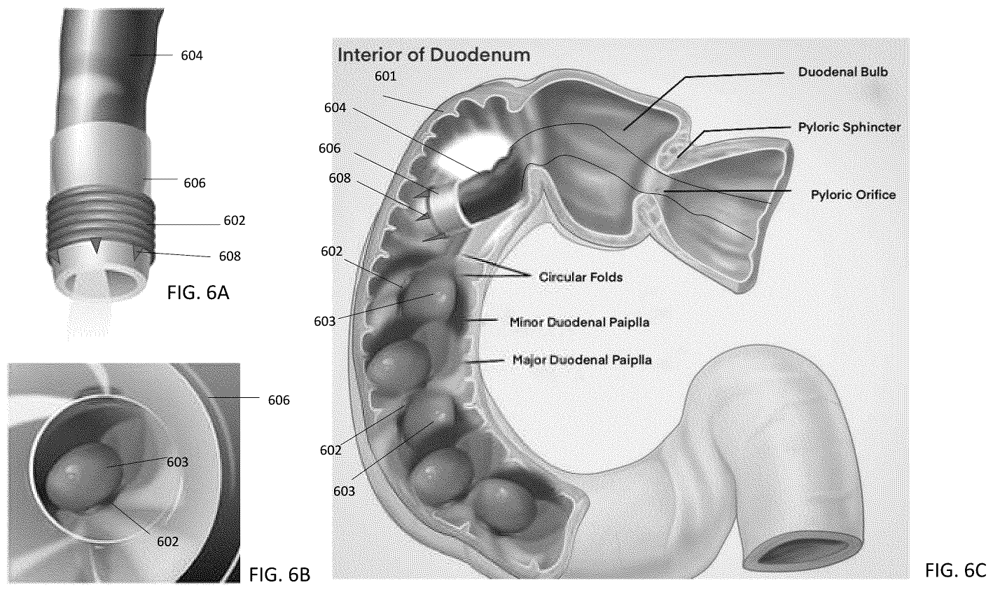

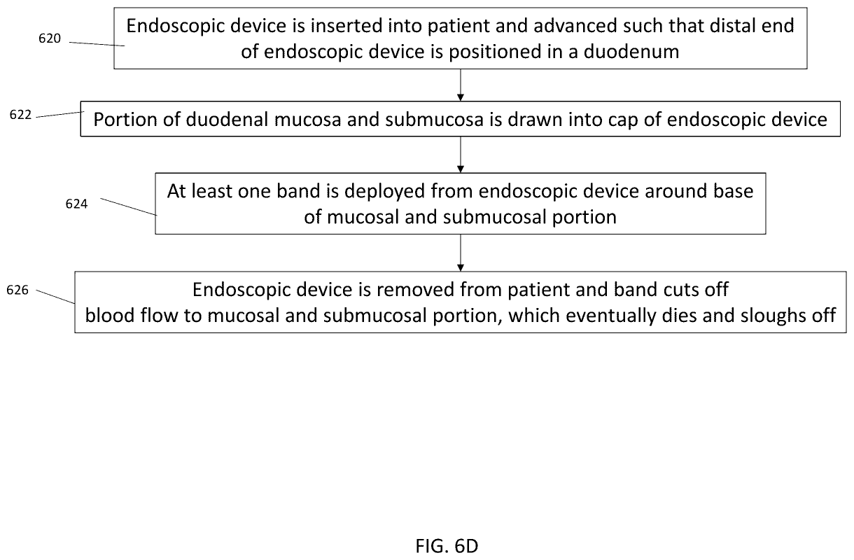

Foregut mucosa resection using a ligation or resection device such as elastic bands is depicted in FIGS. 6A, 6B, and 6C. FIG. 6A illustrates a distal end of an endoscopic device 604 comprising resection components or bands 602 configured for resecting portions of foregut mucosa, in accordance with some embodiments of the present specification. FIG. 6B illustrates a portion 603 of foregut mucosa with a band 602 around a base of the mucosal portion 603 after having been deployed by the device of FIG. 6A. FIG. 6C illustrates a schematic cross-sectional view of a portion of a proximal foregut with the distal end of an endoscopic device 604 positioned within a duodenum 601 and a plurality of foregut mucosa and submucosa portions 603 with bands positioned around bases of the mucosal portions. Although, elastic bands are depicted as a preferred method for resection of the mucosa and/or submucosa, other resection devices and/or techniques known in the art can also be used to resect and/or dissect foregut mucosa and/or submucosa. In all methods of resection, the foregut muscularis propria and adventitia or serosa is left behind functional and intact on which the submucosa and mucosa later regenerate to restore the normal structure and/or function of the targeted foregut.

Referring simultaneously to FIGS. 6A, 6B, and 6C, the endoscopic device 604 comprises a distal end or cap 606 with one or more ligation devices such as bands 602 positioned around the cap 606. In embodiments, the one or more bands 602 are elastic bands that are stretched out when positioned around the cap 606 and then contract once deployed around a section of mucosa. In some embodiments, the endoscopic device is configured to draw a portion of mucosa into the cap 606 and then deploy a band 602 around a base of the mucosa portion 603 drawn into the cap 606. In some embodiments, suction is applied to the endoscopic device 604 to draw the mucosa portion 603 into the cap 606. In some embodiments, the one or more bands 602 include an anchor 608 configured to grasp or pierce the mucosa portion 603 and assist in keeping the band 602 in place after deployment and prevent premature slippage of the band. In some embodiments, the cap 606 is transparent. FIG. 6B illustrates, through a transparent cap 606, a mucosa portion 603 drawn into the cap 606 with a band 602 positioned around the base of the mucosa portion 603. In another embodiment, another resection device or method can be used to resect the foregut mucosa and/or submucosa. In yet another device, a mechanical morcellation device can be used to remove the intestinal mucosa and submucosa.

Referring to FIG. 6C, a plurality of mucosal and submucosal sections 603 with a band 602 around each base of each section is depicted. The elastic properties of the bands 602 causes them to constrict around the base of each mucosa portion 603, effectively cutting off blood supply to the mucosal and submucosal portion 603. Each entrapped portion 603 eventually dies, sloughs off, and is passed by the GI system. Over the next few days to a week or more, the resected area regenerates with improved function and structure resulting in improvement of glucose homeostasis and insulin resistance.

FIG. 6D is a flowchart listing the steps involved in using an endoscopic device to resect or remove tissue, in accordance with one embodiment of the present specification. At step 620, an endoscopic device is inserted into a patient and advanced such that the distal end of the device is positioned in a duodenum of the patient. At step 622, a portion of the duodenal mucosa is drawn into a cap at the distal end of the endoscopic device. At step 624, at least one band is deployed from the distal end of the endoscopic device and positioned around a base of the mucosa portion. The endoscopic device is removed from the patient at step 626, allowing the band to remain and cut off blood flow to the mucosa portion, which eventually dies and sloughs off. Over the next few days to a week or more, the resected area regenerates with improved and/or normal function and/or structure. In another embodiment, a morcellation device passed through the channel of the endoscope can be used to resect/remove intestinal mucosal and/or submucosal tissue.

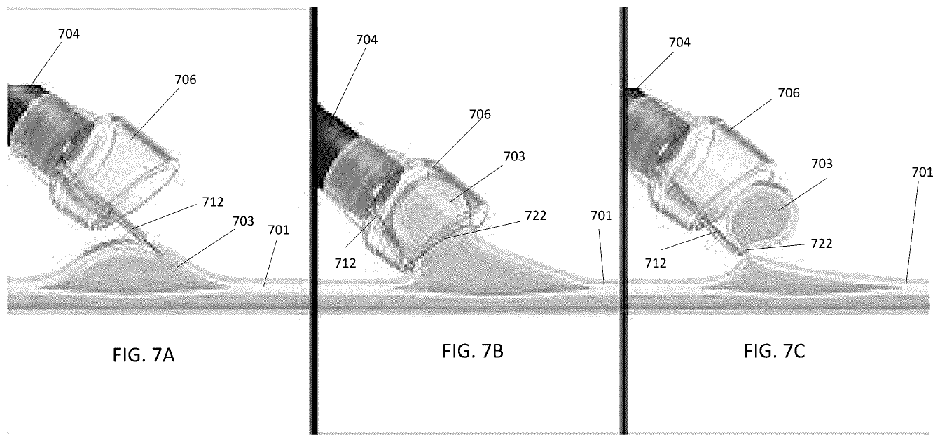

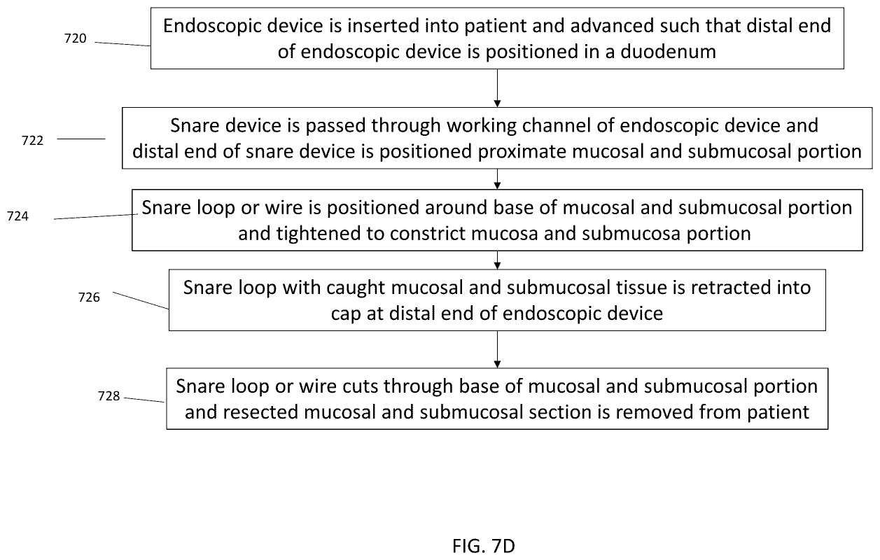

A sequential progress of cap assisted foregut mucosa resection is illustrated in FIGS. 7A, 7B, and 7C. FIG. 7A illustrates a distal end of an endoscopic device 704 for cap assisted mucosal and submucosal resection, in accordance with some embodiments of the present specification. The endoscopic device 704 includes a distal end or cap 706 and is configured to allow a snare device 712 to pass within a working channel of the endoscopic device 704 and extend through and beyond the cap 706. The distal end or cap 706 of the endoscopic device 704 is positioned proximate a target tissue or mucosa portion 703 of a duodenum 701 of a patient. In some embodiments, the cap 706 is transparent. FIG. 7B illustrates the endoscopic device 704 of FIG. 7A with a portion of foregut mucosa 703 pulled into the cap 706 of the device 704. A resection component, comprising a snare loop or wire 722, extends from the snare device 712 and is looped about a base of the mucosa portion 703. In some embodiments, the endoscopic device 704 is configured to draw the portion of mucosa 703 into the cap 706. In some embodiments, suction is applied to the endoscopic device 704 to draw the mucosa portion 703 into the cap 706. Submucosal lifting agent known in the art can be used to assist with tissue resection/removal.

FIG. 7C illustrates the endoscopic device 704 of FIGS. 7A and 7B with the portion of foregut mucosa 703 released from the cap 706 and a snare loop or wire 722 positioned around a base of the mucosal portion 703. The snare loop or wire 722 has been retracted back into the snare device 712 such that the snare loop or wire tightens around or constricts the base of the mucosa portion 703, effectively cutting off blood supply to the mucosa portion 703. In some embodiments, the snare loop or wire 722 is withdrawn completely into the snare device 712, which cuts the mucosa portion off of the wall of the duodenum, resecting the portion of mucosa 703. In some embodiments, electrical current is provided to the snare loop or wire to heat the wire and assist with cutting the tissue (hot snare resection). If electrical current is not provided, the resection is termed a cold snare resection. The resected portion may then be withdrawn from the patient. In other embodiments, a clip may be applied to the constricted base of the mucosa portion 703. The mucosa portion, lacking blood supply, eventually dies and sloughs off. Over the next few days to a week or more, the resected area regenerates with improved function and/or structure.

FIG. 7D is a flowchart listing the steps involved in using an endoscopic device to resect tissue, in accordance with another embodiment of the present specification. At step 720, an endoscopic device is inserted into a patient and advanced such that the distal end of the device is positioned in a duodenum of the patient. At step 722, a snare device is passed through a working channel of the endoscopic device and the distal end of the snare device is positioned proximate the mucosa portion. At step 724, a snare loop or wire is positioned around a base of the mucosa portion and tightened to constrict the mucosal and submucosal tissue. At step 726, the snare loop with caught mucosal and submucosal tissue is optionally retracted into a cap at the distal end of the endoscopic device. At step 728, the snare loop or wire cuts through the base of the mucosa portion and the resected mucosa section is removed from the patient or allowed to pass naturally.

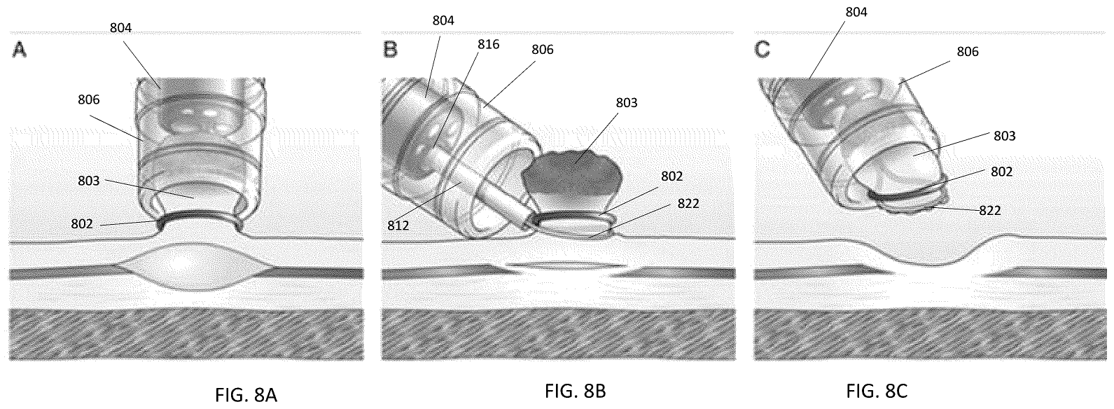

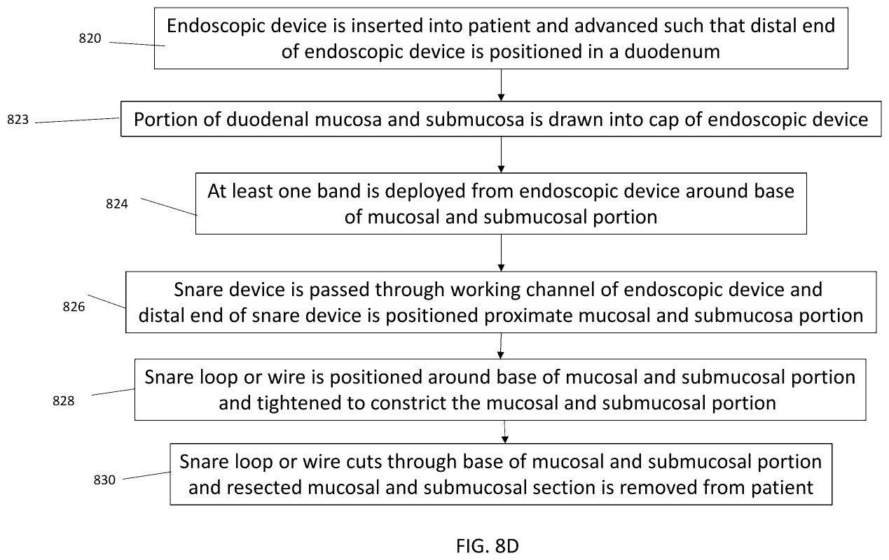

A sequential progress of foregut mucosa resection using a band and a snare is illustrated in FIGS. 8A, 8B, and 8C. FIG. 8A illustrates the distal end or cap 806 of an endoscopic device 804 with a resection component or band 802 positioned around a base of a portion of foregut mucosa 803, in accordance with some embodiments of the present specification. In some embodiments, the endoscopic device 804 is configured to draw the portion of mucosa 803 into the cap 806. In some embodiments, suction is applied to the endoscopic device 804 to draw the mucosa portion 803 into the cap 806. Once deployed, the resection component or band 802, due to its elastic properties, returns to its non-stretched state and constricts around the mucosa portion 803, cutting off blood supply to the mucosal and submucosal tissue 803 and form a polypoidal lesion.

FIG. 8B illustrates the device of FIG. 8A with a snare loop or wire 822 of a snare device 812 positioned around the base of the portion of foregut mucosal and submucosal polypoidal lesion 803, in accordance with some embodiments of the present specification. The endoscopic device 804 is configured to allow the snare device 812 to pass through a working channel 816. In an embodiment, snare loop or wire 822 of the snare device 812 is extended and positioned around a base of the constricted mucosa portion 803. In some embodiments, the snare loop or wire 822 is positioned below the previously placed band 802. The snare loop or wire 822 is drawn back into the snare device 812, causing the snare loop or wire 822 to constrict about the base of the mucosa section 803. In some embodiments, the snare loop or wire 822 is used to constrict and cut the base of the mucosa portion 803, resecting the mucosa portion 803, immediately after band 802 placement. In some embodiments, electrical current is provided to the snare loop or wire to heat the wire and assist with cutting the tissue (hot snare resection). In other embodiments, a band 802 is initially placed and allowed to cut off blood flow to the mucosa portion 803. Over a period of time, due to the pressure applied by the deployed band 802, blood flow to the constricted tissue is obstructed, and the tissue dies gradually. The endoscopic device 804 is then later reinserted into the patient and the snare loop or wire 822 is used to remove the mucosa portion 803, which has died due to lack of blood flow. FIG. 8C illustrates the endoscopic device 804 of FIGS. 8A and 8B, depicting withdrawal of the snare loop or wire 822 along with the resected portion of foregut mucosa 803 with band 802 placed thereupon, in accordance with some embodiments of the present specification.

FIG. 8D is a flowchart listing the steps involved in using an endoscopic device to resect tissue, in accordance with another embodiment of the present specification. At step 820, an endoscopic device is inserted into a patient and advanced such that the distal end of the device is positioned in a duodenum of the patient. At step 823, a portion of the duodenal mucosa is drawn into a cap at the distal end of the endoscopic device. At step 824, at least one band is deployed from the distal end of the endoscopic device and positioned around a base of the mucosa portion. At step 826, a snare device is passed through a working channel of the endoscopic device and the distal end of the snare device is positioned proximate the mucosa portion. At step 828, a snare loop or wire is positioned around a base of the mucosa portion and tightened to constrict the mucosa portion. At step 830, the snare loop or wire cuts through the base of the mucosal and submucosal portion and the resected mucosal and submucosal tissue is removed from the patient.

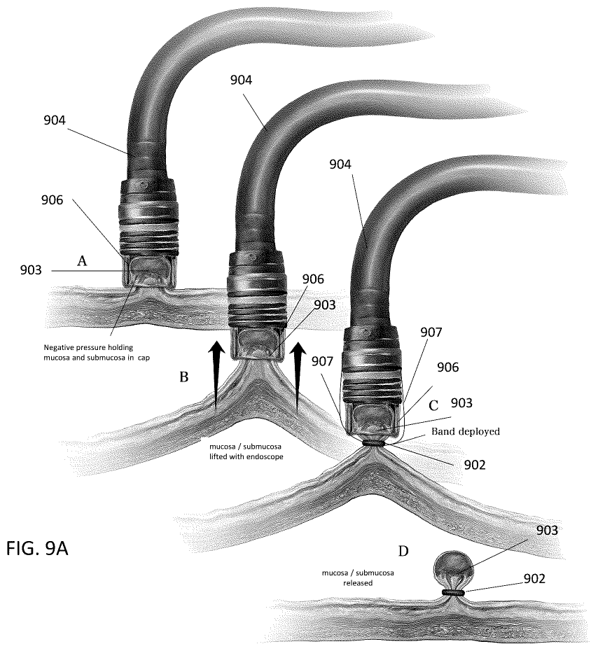

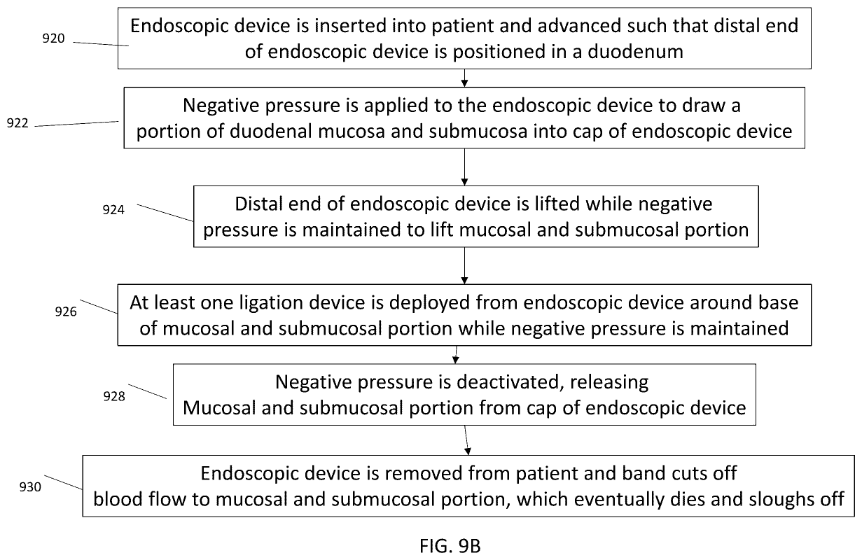

FIG. 9A illustrates an endoscopic device 904 configured to use negative pressure or suction pressure to lift a portion of foregut mucosa 903 and deploy a resection component/ligation band 902, in accordance with some embodiments of the present specification. The endoscopic device includes a cap 906 at its distal end. The endoscopic device 904 is inserted into a patient and maneuvered to the location of target tissue or mucosa portion 903 as illustrated at step A. Negative pressure is applied to endoscopic device 904 to draw a mucosal and submucosal tissue 903 into the cap 906. The negative pressure is maintained to hold the mucosa portion 903 within the cap 906. In some embodiments, the negative pressure within the cap is activated from controls provided at a proximal end of the endoscopic device. At step B, the distal end of the endoscopic device 904 is lifted, while negative pressure is maintained, to lift the mucosa portion 903. At step C, while negative pressure is maintained, a band or ligator 902, comprising one of one or more bands positioned on an outer surface of the cap 906, is deployed and positioned around a base of the mucosal and submucosal tissue 903. In some embodiments, a user may actuate a releasing mechanism of the band from controls provided at a proximal end of the endoscopic device. In some embodiments, a pair of wires 907 extending from the proximal end of the device to diametrically opposite sides of band 902 are used to pull and deploy the band 902 so that band 902 slides off the cap 906 and on to the tissue. The band is released and positioned to form a loop around the base of the mucosa portion 903. At step D, the negative pressure is deactivated so that the mucosa portion 903 is released from the cap 906 while the band constricts the mucosa portion 903. The elastic properties of the band 902 cause it to tighten around the base of the mucosa portion 903, cutting off blood supply to the portion. The mucosal and submucosal tissue 903 eventually dies and sloughs off leaving behind an intact muscularis propria and serosa.

FIG. 9B is a flowchart listing the steps involved in using an endoscopic device to resect tissue, in accordance with another embodiment of the present specification. At step 920, an endoscopic device is inserted into a patient and advanced such that the distal end of the device is positioned in a duodenum of the patient. At step 922, negative pressure is applied to the endoscopic device to draw a portion of the duodenal mucosa into a cap at the distal end of the endoscopic device. At step 924, the distal end of the endoscopic device is lifted while negative pressure is maintained to lift the mucosa portion. At step 926, at least one band is deployed from the distal end of the endoscopic device and positioned around a base of the mucosa portion. At step 928, negative pressure is deactivated, releasing the mucosa portion from the cap of the endoscopic device. The endoscopic device is removed from the patient at step 930, allowing the band or ligation device to remain and cut off blood flow to the mucosal and submucosal tissue, which eventually dies and sloughs off.

In various embodiments of the present specification, the end cap positioned at a distal end of the endoscope may be made of one or more of: polycarbonate, PVC, silicone, or Teflon non-latex rubber material. The end-cap is preferably made of a transparent material to enable visibility of the procedure. In embodiments, the end cap has a diameter in the range of 5 mm to 25 mm. Additionally, in embodiments, the bands configured to be positioned on the end cap are made of one of silicone, Teflon rubber, or non-latex rubber.

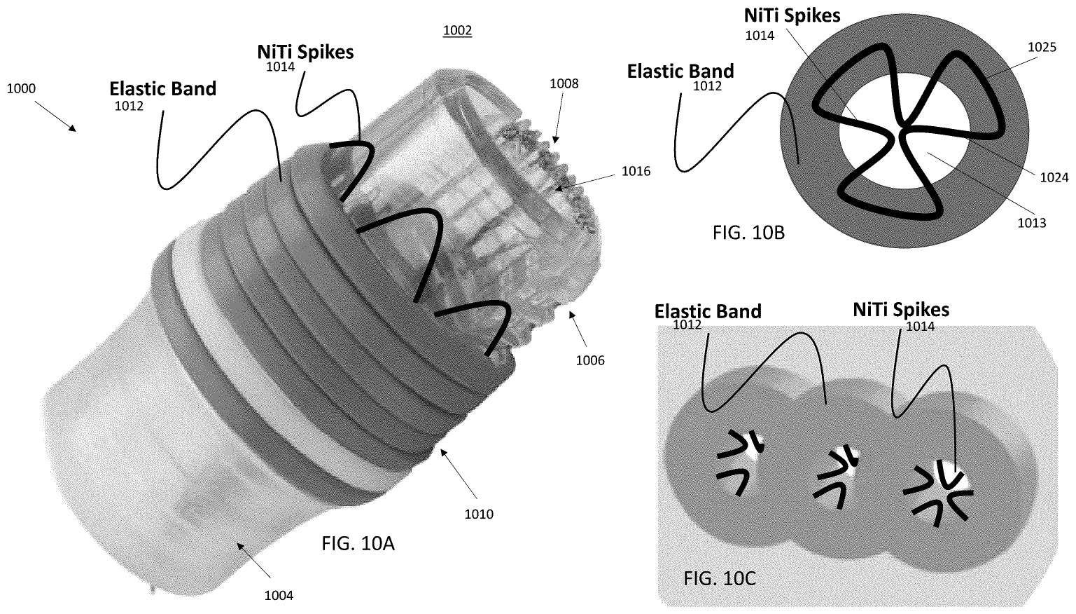

FIG. 10A illustrates a distal end of an endoscopic device 1000 having a plurality of resection components or bands 1012 with shape-memory spikes positioned on the distal end, in accordance with some embodiments of the present specification. The endoscopic device 1000 comprises an endcap 1002 which, in embodiments, is an elongated cylindrical structure having three portions along its longitudinal length. A first proximal portion 1004 is used to attach endcap 1002 to the distal end of an endoscope. A second distal portion 1006 consists of a transparent cylindrical wall with an opening on its distal side. In some embodiments, a portion 1008 of the circumference of the opening is serrated while the remaining circumference is smooth. The serrations house wires 1016 that are positioned along the longitudinal surface of the endcap 1002, and which pull on resection bands 1012, and prevent bands 1012 from sliding (similar to a pully groove) along the circumferential surface of endcap 1002. A third middle portion 1010, which is positioned between and connects the first and third portions 1004 and 1006, comprises one or more elastic resection bands 1012 located around the external surface of the cap 1002. In embodiments, each elastic band 1012 is designed in the form of a doughnut shape, with a hollow inside the cylindrical ring of band 1012. In embodiments, during manufacturing, material of band 1012 is over-molded on to a shape-memory wire such as Nitinol. The combination of band 1012 and wire (wire 1014) provide the required compressive function for resection. Additionally, wire 1014 provides the ability to anchor into the tissue while preventing band 1012 from slipping prematurely allowing for complete ischemic necrosis followed by sloughing of the banded tissue. FIG. 10B illustrates a cross-sectional view of band 1012. Wire 1014 extends through portions of the band 1012 and sections of the wire extend into the center hollow 1013 of the band 1012, through holes that are punctured in the surface of band 1012. In embodiments, wire 1014 is made from a shape-memory alloy, such as, for example Nitinol. In its original, compressed form, wire 1014 forms spikes with peaks 1024 directed toward the hollow center 1013 of band 1012, while the troughs 1026 are within the band 1012. Wire 1014 forms a closed loop. FIG. 10C illustrates a front perspective view of multiple bands 1012 showing spikes of a shape-memory wire 1014 that extends out from a body of each band toward a center of each band, in accordance with some embodiments of the present specification. Referring to FIGS. 10A-10C, the bands 1012, once deployed, are configured to be positioned around, and cut off blood supply from, a section of mucosal tissue. In embodiments, the bands 1012 are stretched into a first configuration and placed on the cap 1002. After deployment, the elastic properties of the bands 1012 causes them to constrict into a second configuration around the base of a mucosa portion. In embodiments, the bands 1012 have a first diameter when in the first configuration and a second diameter when in the second configuration. In embodiments, the first diameter is greater than the second diameter. The wire 1014 within the band 1012 is configured to change shape from a first configuration when the band 1012 is positioned on the cap 1002 to a second configuration when the band is deployed on a mucosa portion. When in the first configuration, the wire 1014 is shaped and adequately sized such that it allows for placement of the band 1012 on the cap 1002 in the maximum expanded position of the band. In other words, the wire 1014, when in the first configuration, has a circular shape that approximates the shape of the stretched band in the first configuration. When the wire 1014 is in the second configuration, the shape changes to comprise peaks or spikes 1024 which protrude into, grasp, or pierce the mucosa portion to assist in holding the band 1012 around the tissue. Additionally, the wire is shaped so that in the second configuration it allows for the band to maximally constrict without significantly interfering with the process of constriction.

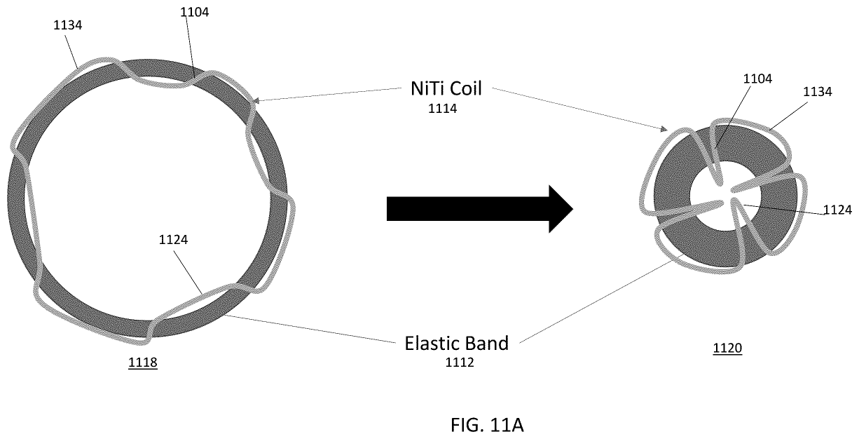

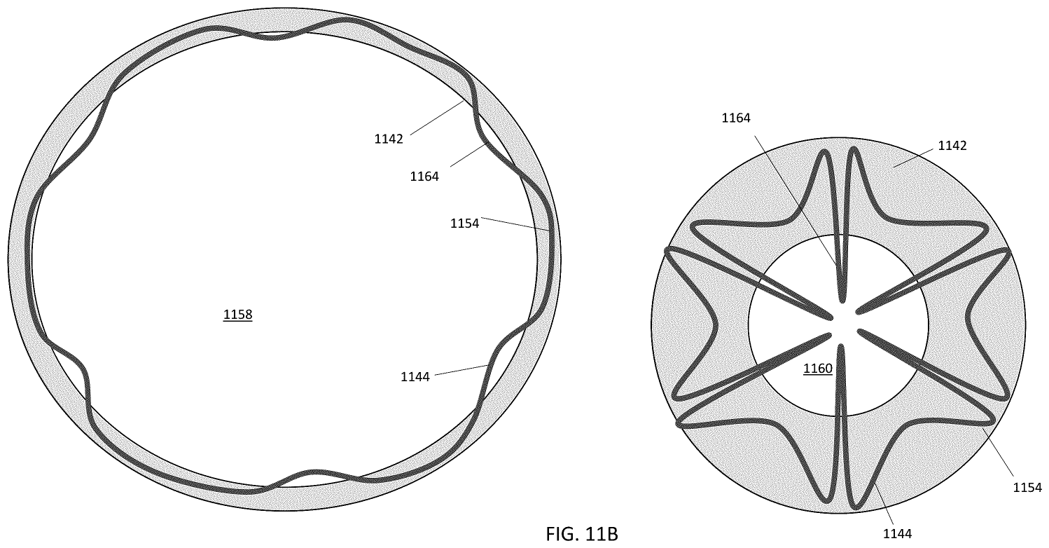

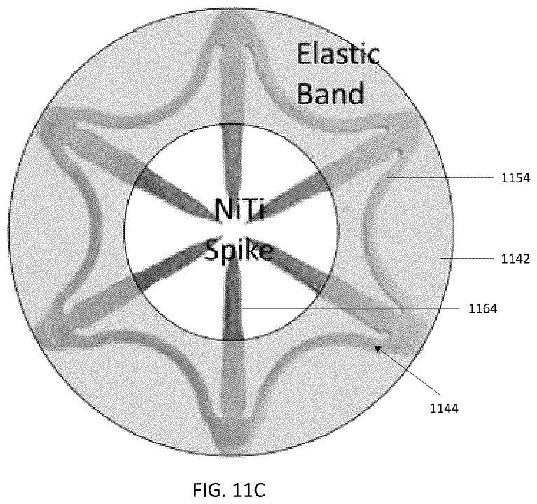

FIG. 11A illustrates a resection component or band 1112 with a shape-memory wire 1114 positioned inside the band in expanded and contracted configurations, in accordance with some embodiments of the present specification. In some embodiments, band 1112 has a first diameter in a range of 1 mm to 5 mm and first thickness in a range of 1 mm to 5 mm in its contracted state and has a second diameter in a range of 5 mm to 25 mm and a second thickness in a range of 0.5 mm to 2.5 mm in its expanded state. The wire 1114 is positioned inside the band 1112 such that first wire portions 1104 are within and contact the material of the band 1112, second wire portions 1124 extend from the band 1112 and toward a center of the band 1112, and third wire portions 1134 extend from the band and away from a center of the band. In a first configuration, depicted in view 1118, the band 1112 is stretched out for placement on a cap of an endoscopic device. The band 1112, in the first configuration, is in a circular shape with a first diameter. The wire 1114, in the first configuration, approximates the shape of the band 1112. After deployment, the band 1112 changes from the first configuration in view 1118 to a second configuration depicted in view 1120. In the second configuration, the elastic properties of the band 1112 have caused it to constrict. The band 1112, in the second configuration, has a circular shape and a second diameter. In embodiments, the second diameter is less than the first diameter. In the second configuration, the second wire portions 1124 form spikes or protrusions that are configured to grasp or pierce the mucosa portion encircled by the band 1112. The second wire portions 1124, or spikes, in the second configuration, assist in keeping the band 1112 placed on the mucosa portion. In some embodiments, the shape-changing properties of the wire 1114 assist in contracting the band 1112. Additionally, the spikes in the pre-deployment runs mostly perpendicular to the inner diameter of the band and in the post-deployment position, predominantly parallel to the inner diameter of the band.

FIG. 11B illustrates a resection component or band 1142 with a shape-memory wire 1144 positioned inside the band in expanded and contracted configurations, in accordance with other embodiments of the present specification. FIG. 11C illustrates a band with a shape-memory wire positioned inside the band in a contracted configuration, in accordance with some embodiments of the present specification. Referring to FIGS. 11B and 11C simultaneously, the wire 1144 is positioned inside the band 1142 such that first wire portions 1154 are within and contact the material of the band 1142 and second wire portions 1164 extend from the band and toward a center of the band. In a first configuration, depicted in view 1158, the band 1142 is stretched out for placement on a cap of an endoscopic device. The band 1142, in the first configuration, is in a circular shape with a first diameter. The wire 1144, in the first configuration, approximates the shape of the band 1142. After deployment, the band 1142 changes from the first configuration in view 1158 to a second configuration depicted in view 1160. In the second configuration, the elastic properties of the band 1142 have caused it to constrict. The band 1142, in the second configuration, has a circular shape and a second diameter. In embodiments, the second diameter is less than the first diameter. In the second configuration, the second wire portions 1164 form spikes or protrusions that are configured to grasp or pierce the mucosa portion encircled by the band 1142. The second wire portions 1164, or spikes, in the second configuration, act as anchors and assist in keeping the band 1142 placed on the mucosa portion, thus preventing band 1142 from prematurely dislodging. In some embodiments, the shape changing properties of the wire 1144 assist in contracting the band 1142.

In different embodiments, including those illustrated by FIGS. 11A-11C, during the compressed state of the shape memory material, a band comprising the shape memory material is forced to compress. The band is compressed around the mucosa and submucosal portion, or the pseudopolyp supported by the shape memory elements of the embodiments in accordance with the present specification. Compression of the band facilitates anchoring of the band in the mucosal tissue, around the pseudopolyp, for resection purposes. The shape memory material prevents the elastic material of the band surrounding the tissue from prematurely dislodging.

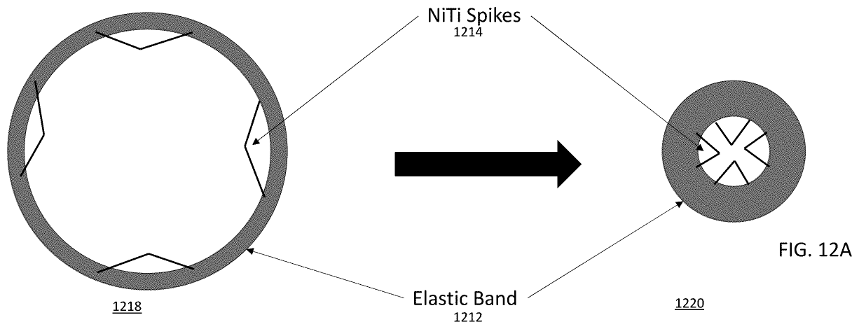

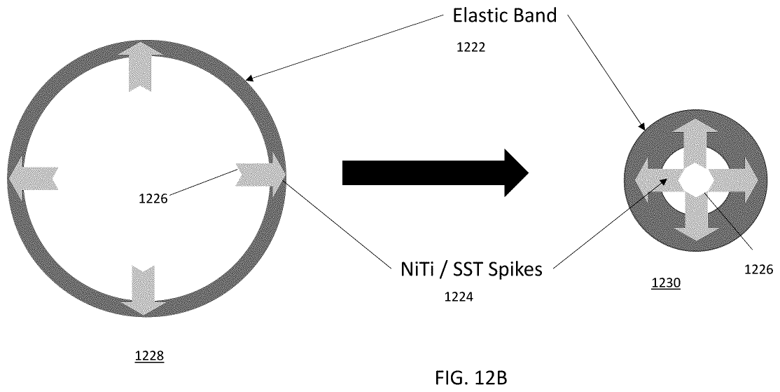

FIG. 12A illustrates a resection component or band 1212 with a plurality of shape-memory wire portions 1214 positioned along an inner circumference of the band 1212 in expanded and contracted configurations, in accordance with some embodiments of the present specification. In embodiments, the wire portions 1214 are positioned along the inner circumference of the band 1212 as protrusions and extend from the band and toward a center of the band. Wire portions 1214 are attached, glued, or over-molded within band 1212 so that the expansion and contraction of the two structures are almost identical. In embodiments, spikes of wire 1214 may be all connected as one structure, or may include multiple disconnected spikes. In a first configuration, depicted in view 1218, the band 1212 is stretched out for placement on a cap of an endoscopic device. The band 1212, in the first configuration, is in a circular shape with a first diameter. In embodiments, the wire portions 1214, in the first configuration, are hill-shaped. In embodiments, the band 1212 includes a range of 1 to 10 wire portions 1214. In one embodiment, the band 1212 includes four wire portions 1214. After deployment, the band 1212 changes from the first configuration in view 1218 to a second configuration depicted in view 1220. In the second configuration, the elastic properties of the band 1212 have caused it to constrict. The band 1212, in the second configuration, has a circular shape and a second diameter. In embodiments, the second diameter is less than the first diameter. In the second configuration, the wire portions 1214 form spikes that are configured to grasp or pierce the mucosa portion encircled by the band 1212. The wire portions 1214, or spikes, in the second configuration, act as anchors and assist in keeping the band 1212 placed on the mucosa portion, thus preventing band 1212 from prematurely dislodging. In some embodiments, the shape changing properties of the wire 1214 assist in contracting the band 1212.

FIG. 12B illustrates a resection component or band 1222 with a plurality of shape-memory or stainless steel wire extensions or spikes 1224 positioned along an inner circumference of the band 1222 in expanded and contracted configurations, in accordance with some embodiments of the present specification. In embodiments, the wire extensions 1224 are positioned along the inner circumference of the band 1222 as protrusions and extend from the band and toward a center of the band. In a first configuration, depicted in view 1228, the band 1222 is stretched out for placement on a cap of an endoscopic device. The band 1222, in the first configuration, is in a circular shape with a first diameter. In embodiments, the wire extensions 1224, in the first configuration, are tower or cylinder-shaped. In some embodiments, the wire extensions 1224 include spiked or serrated distal ends 1226. In embodiments, the band 1222 includes a range of 1 to 10 wire extensions 1224. In one embodiment, the band 1222 includes four wire extensions 1224. After deployment, the band 1222 changes from the first configuration in view 1228 to a second configuration depicted in view 1230. In the second configuration, the elastic properties of the band 1222 have caused it to constrict. The band 1222, in the second configuration, has a circular shape and a second diameter. In embodiments, the second diameter is less than the first diameter. In the second configuration, the wire extensions 1224 form spikes that are configured to grasp or pierce the mucosa portion encircled by the band 1222. The wire extensions 1224, or spikes, in the second configuration, act as anchors and assist in keeping the band 1222 placed on the mucosa portion, thus preventing band 1222 from prematurely dislodging.

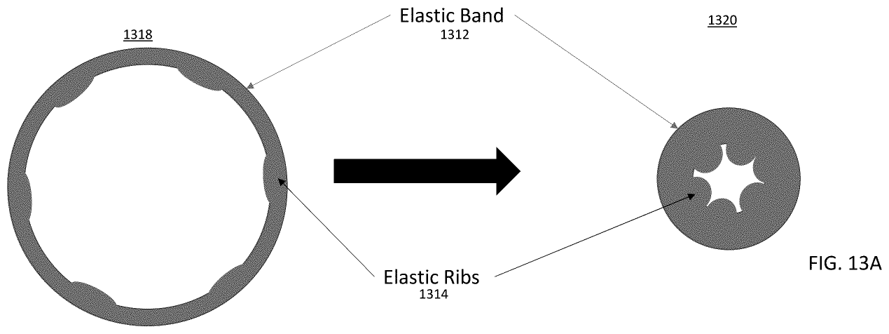

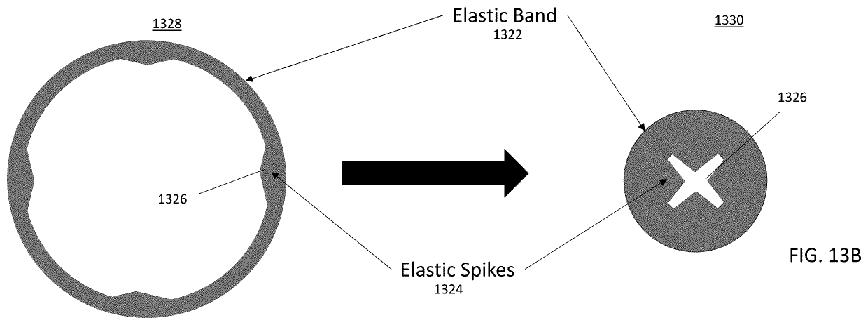

FIG. 13A illustrates a resection component or band 1312 with a plurality of protrusions 1314 extending toward a center of the band in expanded and contracted configurations, in accordance with some embodiments of the present specification. In embodiments, the protrusions 1314 are part of the elastic band 1312, made from same or different material as the band and are positioned along the inner circumference of the band 1312, extending from the band and toward a center of the band. In a first configuration, depicted in view 1318, the band 1312 is stretched out for placement on a cap of an endoscopic device. The band 1312, in the first configuration, is in a circular shape with a first diameter. In embodiments, the protrusions 1314, in the first configuration, are hill-shaped with a first base diameter and a first height. In embodiments, the band 1312 includes a range of 1 to 10 protrusions 1314. In one embodiment, the band 1312 includes four protrusions 1314. After deployment, the band 1312 changes from the first configuration in view 1318 to a second configuration depicted in view 1320. In the second configuration, the elastic properties of the band 1312 have caused it to constrict. The band 1312, in the second configuration, has a circular shape and a second diameter. In embodiments, the second diameter is less than the first diameter. In the second configuration, in embodiments, the protrusions 1314 are hill-shaped with a second base diameter and a second height. In embodiments, the second base diameter is less than the first base diameter and the second height is greater than the first height. The protrusions 1314, in the second configuration, are configured to grasp or pierce the mucosa portion encircled by the band 1312. The protrusions 1314, in the second configuration, act as anchors and assist in keeping the band 1312 placed on the mucosa portion, thus preventing band 1312 from prematurely dislodging. Other band surface architecture features can be incorporated in the band to increase the roughness of the band surface and preventing it from slipping off the mucosa prematurely.

FIG. 13B illustrates a resection component or band 1322 with a plurality of protrusions 1324 extending toward a center of the band in expanded and contracted configurations, in accordance with other embodiments of the present specification. In embodiments, the protrusions 1324 are part of the band 1322 and are positioned along the inner circumference of the band 1322, extending from the band and toward a center of the band. In a first configuration, depicted in view 1328, the band 1322 is stretched out for placement on a cap of an endoscopic device. The band 1322, in the first configuration, is in a circular shape with a first diameter. In embodiments, the protrusions 1324, in the first configuration, are cone-shaped with a pointed distal end 1326, with a first base diameter and a first height. In embodiments, the band 1322 includes a range of 1 to 10 protrusions 1324. In one embodiment, the band 1322 includes four protrusions 1324. After deployment, the band 1322 changes from the first configuration in view 1328 to a second configuration depicted in view 1330. In the second configuration, the elastic properties of the band 1322 have caused it to constrict. The band 1322, in the second configuration, has a circular shape and a second diameter. In embodiments, the second diameter is less than the first diameter. In the second configuration, in embodiments, the protrusions 1324 are cone-shaped with a second base diameter and a second height. In embodiments, the second base diameter is less than the first base diameter and the second height is greater than the first height. The protrusions 1324, in the second configuration, are configured to grasp or pierce the mucosa portion encircled by the band 1312. The protrusions 1324, in the second configuration, act as anchors and assist in keeping the band 1322 placed on the mucosa portion, thus preventing band 1322 from prematurely dislodging. In another embodiment, grooves on the surface of the band increases its anchoring capability and prevent premature dislodgement.

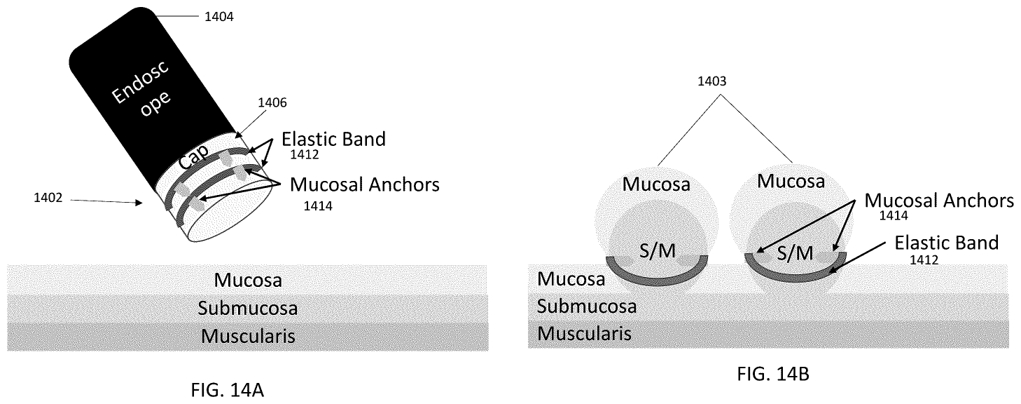

FIG. 14A illustrates distal end 1402 of an endoscope 1404 comprising a cylindrical cap 1406 with at least two resection components or elastic bands 1412 positioned around an external surface of cap 1406, in accordance with some embodiments of the present specification. In embodiments, one or more elastic bands 1412 are positioned around cap 1406 before the bands are deployed for resection of mucosal tissue. Anchors 1414, such as a coil with spikes or individual spikes made from a shape-memory material such as Nitinol, are aligned along the length of band 1412 before deployment of band 1412. Each band 1412 has a corresponding set of anchors 1414 positioned on it. FIG. 14B illustrates bands 1412 of device FIG. 14A deployed around bases of foregut mucosal portions 1403. Post-deployment, anchors 1414 are deployed inwards toward the center of the band to grasp or pierce the mucosal portions 1403 to enable anchoring into mucosal tissue. Blood flow to the mucosal and submucosal tissue 1403 is cut off and, over a period of time, the mucosal and submucosal portions of the intestine undergo ischemic necrosis and slough off.

In general, it is preferred for the aforementioned resection components or bands to be made of a first material and the anchors to be made a second material where the first material is different from the second material. More preferably, the first material has a greater elasticity than the second material, and/or the first material has a shore durometer value that is less than the shore durometer value of the second material. More preferably, if the circular band is cut and laid out linearly, its entire length is equal to a first value and if the anchor is similarly cut and/or laid out linearly, its entire length is equal to a second value where the second value is greater than the first value. More preferably, a portion of the anchor is physically attached or embedded into the band, where that portion has a length that is equal to or less than 70%, preferably equal to or less than 50% and more preferably equal to or less than 25%, of the entire length of the anchor. More preferably, the unstretched band has a diameter equal to a first value and the anchor has one or more protrusions extending from the inner surface of the band, wherein each protrusion extends a distance into the area encircled by the band and wherein the distance is equal to at least 5% of the diameter, at least 10% of the diameter, at least 20% of the diameter, at least 30% of the diameter, at least 40% of the diameter, at least 50% of the diameter, at least 60% of the diameter, at least 70% of the diameter, at least 80% of the diameter, or at least 90% of the diameter. More preferably, the unstretched band has a diameter equal to a first value and the anchor has one or more protrusions extending from the inner surface of the band, wherein each protrusion extends a distance into the area encircled by the band and wherein the distance is equal to no more than 50% of the diameter, no more than 40% of the diameter, no more than 30% of the diameter, no more than 20% of the diameter, no more than 10% of the diameter, or no more than 5% of the diameter. The band may be divided into two equal halves, wherein the anchor comprises two protrusions where each of the two protrusions is positioned centrally within, and extends out from, each of the two halves. The band may be divided into three equal thirds, wherein the anchor comprises three protrusions where each of the three protrusions is positioned centrally within, and extends out from, each of the three thirds. The band may be divided into four equal fourths, wherein the anchor comprises four protrusions where each of the four protrusions is positioned centrally within, and extends out from, each of the four fourths. The band may be divided into five equal fifths, wherein the anchor comprises five protrusions where each of the five protrusions is positioned centrally within, and extends out from, each of the five fifths. The band may be divided into six equal sixths, wherein the anchor comprises six protrusions where each of the six protrusions is positioned centrally within, and extends out from, each of the six sixths. The band may be divided into seven equal sevenths, wherein the anchor comprises seven protrusions where each of the seven protrusions is positioned centrally within, and extends out from, each of the seven sevenths. The band may be divided into eight equal eighths, wherein the anchor comprises eight protrusions where each of the eight protrusions is positioned centrally within, and extends out from, each of the eight eighths. The band may be divided into nine equal ninths, wherein the anchor comprises nine protrusions where each of the nine protrusions is positioned centrally within, and extends out from, each of the nine ninths. The band may be divided into ten equal tenths, wherein the anchor comprises ten protrusions where each of the ten protrusions is positioned centrally within, and extends out from, each of the ten tenths.

In various embodiments, more than five discrete and less than 100 discrete resection components or elastic bands are applied to the foregut mucosa and left in place to resect the foregut mucosa or submucosa over a period of time using ischemic necrosis of the mucosa. In some embodiments, the bands are deployed and left in their position for approximately seven days. In some embodiments, the bands are deployed and left in their position for approximately one day. In another embodiment, the bands are deployed and left in their position for approximately 30 days. In most embodiments, the bands slough-off without the need for additional intervention after the ischemic necrosis process is completed and are naturally passed out of the human body.

In other embodiments, more than five discrete and less than 100 discrete resection components or bands are applied to the foregut mucosa and the foregut mucosa or submucosa is resected during the endoscopic procedure using hot or cold snare resection or band resection technique. In yet another embodiment, more than five discrete and less than 100 discrete areas of foregut mucosa and the foregut mucosa or submucosa are resected during the endoscopic procedure using cap assisted hot or cold snare resection technique.

In yet another embodiment, duodenal periampullary mucosa or submucosa are resected without resecting an ampullary mucosa during the procedure using cap assisted hot or cold snare or band resection technique.

In yet another embodiment, the foregut mucosa and/or submucosa is resected using endoscopic submucosal dissection technique where the foregut mucosa and/or submucosa is resected in one or more non-contiguous sections wherein the cumulative surface area of the resected mucosa is between 5 cm² and 100 cm².

In one embodiment, between 5 cm² and 500 cm² of proximal foregut mucosa is resected. In another embodiment, non-contiguous portions of the foregut mucosa are resected using multiple resection components or bands and corresponding anchor configurations. In cases of non-contiguous resection, a single mucosal resection area is less than 5 cm² and the cumulative mucosal resection area may be greater than 10 cm². In another embodiment, less than 50% of a contiguous circumferential mucosa is resected while greater than 50% non-contiguous circumferential mucosa is resected. In another embodiment, less than 5 cm of contiguous length of foregut mucosa is resected while greater than 5 cm of cumulative length of foregut mucosa is resected. In yet another embodiment, the resection is performed in two or more discrete sections of the foregut mucosa or submucosa, wherein the resected mucosa or submucosa is intervened by one or more sections of non-resected mucosa or submucosa. In some embodiments, the intervening non-ligated and/or resected mucosa can be treated with a different ablation or electroporation or morcellation technology to maximize the treated surface area.

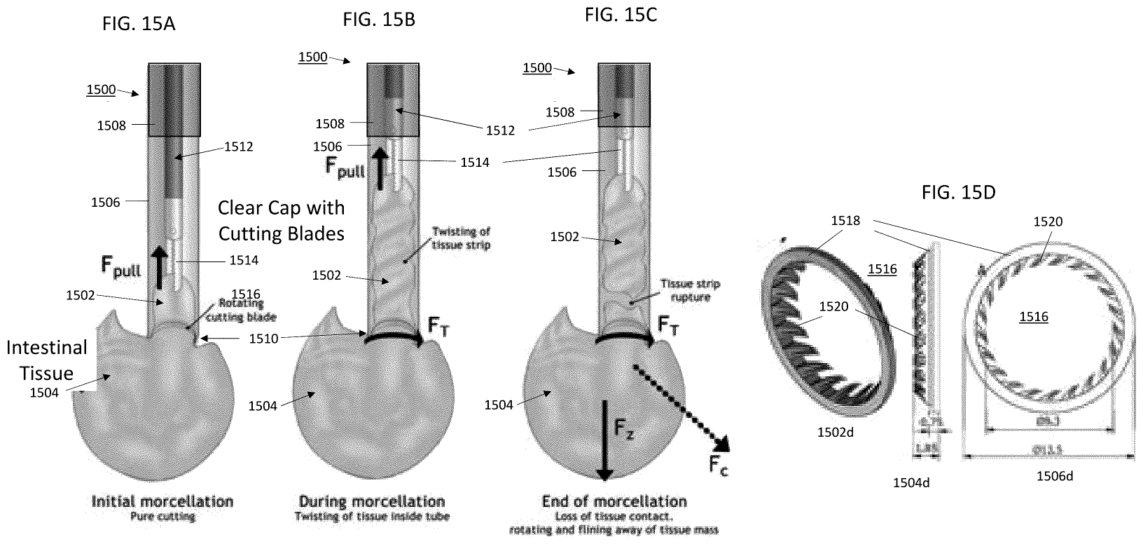

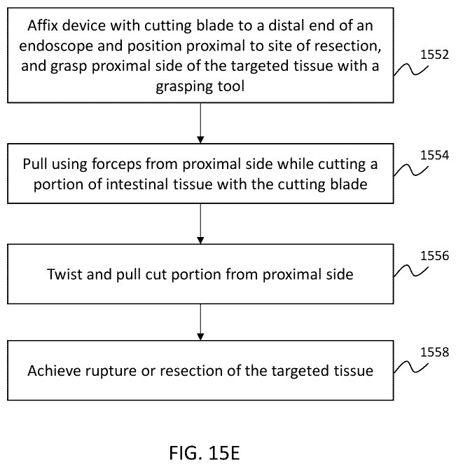

FIGS. 15A to 15C illustrate use of a device comprising an endoscope with a cutting tool in a series of steps to achieve morcellation for removal of resected tissue from a duodenal mucosal and/or submucosal surface. FIG. 15D illustrates a top side perspective view 1502d, a side view 1504d, and a top view 1506d of a rotating cutting blade 1516, used with the cutting tool of FIGS. to 15C, in accordance with some embodiments of the present specification. FIG. 15E is a flow chart illustrating an exemplary set of steps used to achieve morcellation using the embodiments of FIGS. 15A to 15D, in accordance with some embodiments of the present specification.

Referring simultaneously to FIGS. 15A to 15E, endoscope 1500 is inserted such that a distal end 1508 of the endoscope, with an attached cap 1506, is positioned proximal to surface of resected tissue 1502. Resected intestinal tissue 1502 is resected using one of the plurality of embodiments of a resection device described here, which causes tissue 1502 to protrude outwards from an intestinal tissue surface 1504. However, resected tissue 1502 may still be attached to intestinal tissue surface 1504 at a site 1510. A rotating cutting blade 1516 which is part of the cap 1506 is positioned to encircle site 1510. Endoscope 1500 includes an elongated cylindrical cap 1506 attached to a distal end 1508 of endoscope 1500. In some embodiments, cap 1506 is made of a transparent material. In embodiments, the cap 1506 comprises a clear material, such as, polycarbonate, PVC, silicone, or any other similar material, and has a diameter ranging from 5 mm to 25 mm and total length ranging from 5 mm to 25 mm. Endoscope 1500 is positioned so that distal circular end of cap 1506 is positioned to encompass site 1510 to be resected, therefore resulting in encasing of targeted tissue 1502 within cap 1506. A grasping tool 1512 is inserted through a lumen within endoscope 1500 so as to enable exit of distal end of tool 1512 through distal end 1508 of endoscope 1500. Tool 1512 is further extended through elongated cap 1506. Attached to a distal end of tool 1512 is a pair of grasping jaws 1514. In some embodiments, grasping jaws 1514 include two linear arms with rectangular surfaces, each arm comprising an internal surface facing the other arm, and an external surface opposite to the internal surface. Each internal surface of each arm may include a corrugated surface to allow for friction and/or grip while grasping a proximal side of ligated tissue 1502 with the arms. At step 1552, rotating cutting blade 1516 is positioned proximal to site 1510 of resection, and jaws 1514 are positioned to grasp proximal side of ligated tissue with the arms of forceps 1514.

At step 1554, and as illustrated by FIG. 15A, rotating cutting blade 1516 is rotated to cut at site 1510 while simultaneously a first force (F_{Pull}) is applied to pull tool 1512 along with the attached jaws 1514 that have a grasp of proximal side of resected tissue 1502, through the lumen of endoscope device 1500 in a direction towards the proximal side. In some embodiments, the cutting blade 1516 rotates to cut tissue. In other embodiments, the grasping tool 1512 is rotated to move the tissue relative to the cutting blade 1516 to cut tissue. In other embodiments, both the cutting blade 1516 and grasping tool 1512 are rotated to cut tissue. In embodiments, the forces applied are applied manually a user. In other embodiments, forces are applied mechanically or automatically by a robotic system. In other embodiments, forces are applied by any combination of manually by a user and mechanically/automatically by a robotic system. Rotating cutting blade 1516 has two sections—a circular ring 1518 with internal surface of diameter 5-25 mm and an external surface of diameter 5-25 mm, and a series of equally spaced cutting blades 1520 that are positioned on the internal surface. Blades 1520 may comprise triangular structures with one base side of each triangle attached to the internal surface of ring 1518, and a pointed side opposite to the base side pointing towards a center of ring 1518. In some embodiments, each blade has a length or distance at a base ranging from 1 to 10 mm and a height ranging from 1 to 5 mm. In some embodiments, each blade has a length or distance at a base of 5 mm or less and a height of 2 mm. In embodiments, each blade 1520 is configured at an angle to the internal surface of ring 1518, so that all blades 1520 appear to point in a clockwise or an anti-clockwise direction when ring 1518 is viewed from a top side. Other blade configurations known in the art can be used for this embodiment.

At step 1556, and as illustrated by FIG. 15B, resected tissue 1502 is twisted within cap 1506 by applying a rotational second force (F T) by tool 1512 and therefore by jaws 1514 that are attached to tool 1512 and have a grasp of resected tissue 1502. Second force F T is applied in addition to the previous application of first force F_{Pull}. Simultaneous action of rotating cutting blade 1516, first force F_{Pull} and second force F_{T}, enable, at step 1558, achieving resection by rupture and consequent separation of resected tissue 1502 from intestinal tissue surface 1504 from a site proximal to site 1510 of resection. FIG. 15C depicts the endoscope 1500 at the end of morcellation, with the cut intestinal tissue 1502 within cap 1506, in accordance with some embodiments of the present specification. Resected and separated mass of tissue 1502 is subsequently pulled out by tool 1512 through proximal side of lumen of endoscopic device 1500. In certain embodiments, the cutting blade 1516 are fixed and the rotational action is provided by the tool 1512. The motion of both the cutting blade 1516 or tool 512 could be vibratory or oscillatory. Referring to FIGS. 15A-15C simultaneously, in FIG. 15A, morcellation is initiated wherein tissue is pulled into the cap (or morcellation tube) 1506, defined as Fₚᵤₗₗ, and a tissue strip is being cut properly. In FIG. 15B, midway through morcellating a tissue strip, the strip has come to be of such length that twisting of the strip inside the cap 1506 occurs. This results in a torque (FT) of the tissue mass, induced by the rotating cutting blade, spinning the tissue. In FIG. 15C, the tissue mass is free to follow the torque FT as well as disconnect from the cap (force Fz), resulting in a combined force vector Fc, indicating the direction to where the tissue mass falls or is flung.

Embodiments of the present specification can be used for treating at least one of excess weight, obesity, eating disorders, metabolic syndrome, dyslipidemia, diabetes, polycystic ovarian disease, fatty liver disease, non-alcoholic fatty liver disease, or non-alcoholic steatohepatitis disease by resecting foregut tissue using a foregut mucosal and/or submucosal resection and/or dissection system.

In various embodiments, resection therapy is provided to achieve the following therapeutic goals or endpoints for patients with obesity, excess weight, eating disorders, dyslipidemia, or diabetes and a first phase of treatment is considered successful for these patients if any one or more of the following therapeutic goals or endpoints is reached: a total body weight of the patient decreases by at least 1% relative to a total body weight of the patient before resection; an excess body weight of the patient decreases by at least 1% relative to an excess body weight of the patient before resection; a total body weight of the patient decreases by at least 1% relative to a total body weight of the patient before resection and a well-being level of the patient does not decrease more than 5% relative to a well-being level of the patient before resection; an excess body weight of the patient decreases by at least 1% relative to an excess body weight of the patient before resection and a well-being level of the patient does not decrease more than 5% relative to a well-being level of the patient before resection; a pre-prandial ghrelin level of the patient decreases by at least 1% relative to a pre-prandial ghrelin level of the patient before resection; a post-prandial ghrelin level of the patient decreases by at least 1% relative to a post-prandial ghrelin level of the patient before resection; an exercise output of the patient increases by at least 1% relative to an exercise output of the patient before resection; a glucagon-like peptide-1 level of the patient increases by at least 1% relative to a glucagon-like peptide-1 level of the patient before resection; a leptin level of the patient increases by at least 1% relative to a leptin level of the patient before resection; the patient's appetite decreases, over a predefined period of time, relative to the patient's appetite before resection; a peptide YY level of the patient increases by at least 1% relative to a peptide YY level of the patient before resection; a lipopolysaccharide level of the patient decreases by at least 1% relative to a lipopolysaccharide level of the patient before resection; a motilin-related peptide level of the patient decreases by at least 1% relative to a motilin-related peptide level of the patient before resection; a cholecystokinin level of the patient increases by at least 1% relative to a cholecystokinin level of the patient before resection; a resting metabolic rate of the patient increases by at least 1% relative to a resting metabolic rate of the patient before resection; a plasma-beta endorphin level of the patient increases by at least 1% relative to a plasma-beta endorphin level of the patient before resection; an HbA1c level of the patient decreases by at least 0.3% relative to an HbA1c level of the patient before resection; a triglyceride level of the patient decreases by at least 1% relative to a triglyceride level of the patient before resection; a total blood cholesterol level of the patient decreases by at least 1% relative to a total blood cholesterol level of the patient before resection; a glycemia level of the patient decreases by at least 1% relative to a glycemia level of the patient before resection; a composition of the person's gut microbiota modulates from a first state before resection to a second state after resection, wherein the first state has a first level of bacteroidetes and a first level of firmicutes, wherein the second state has a second level of bacteroidetes and a second level of firmicutes, wherein the second level of bacteroidetes is greater than the first level of bacteroidetes by at least 3%, and wherein the second level of firmicutes is less than the first level of firmicutes by at least 3%; or, a cumulative daily dose of the patient's antidiabetic medications decreases by at least 10% relative to a cumulative daily dose of the patient's antidiabetic medications before resection. In most embodiments, the incidence of severe hypoglycemia is decreased to <1%.

In various embodiments, resection therapy is provided to achieve the following therapeutic goals or endpoints for patients with dyslipidemia and a first phase of treatment is considered successful for these patients if any one or more of the following therapeutic goals or endpoints is reached: a lipid profile of the patient improves by at least 10% relative a lipid profile of the patient before resection, wherein lipid profile is defined at least by a ratio of LDL cholesterol to HDL cholesterol, and improve is defined as a decrease in the ratio of LDL cholesterol to HDL cholesterol; an LDL-cholesterol level of the patient decreases by at least 10% relative to an LDL-cholesterol level of the patient before resection; or, a VLDL-cholesterol level of the patient decreases by at least 10% relative to a VLDL-cholesterol level of the patient before resection.

In various embodiments, resection therapy is provided to achieve the following therapeutic goals or endpoints for patients with non-alcoholic steatohepatitis (NASH) or non-alcoholic fatty liver disease (NAFLD), and a first phase of treatment is considered successful for these patients if any one or more of the following therapeutic goals or endpoints is reached: at least a 10% decrease in either ALT or AST levels relative to ALT or AST levels before resection; at least a 10% improvement in serum ferritin level or an absolute serum ferritin level of less than 1.5 ULN (upper limit normal) relative to serum ferritin levels before resection; at least a 5% improvement in hepatic steatosis (HS) or less than 5% HS relative to HS levels before resection, as measured on liver biopsy; at least a 5% improvement in HS or less than 5% HS relative to HS levels before resection, as measured by magnetic resonance (MR) imaging, either by spectroscopy or proton density fat fraction; at least a 5% improvement in an NAFLD Fibrosis Score (NFS) relative to an NFS before resection; at least a 5% improvement in an NAFLD Activity Score (NAS) relative to an NAS before resection; at least a 5% improvement in a Steatosis Activity Fibrosis (SAF) score relative to an SAF score before resection; at least a 5% decrease in a mean annual fibrosis progression rate relative to a mean annual fibrosis progression rate before resection, as measured by histology, Fibrosis-4 (FIB-4) index, aspartate aminotransferase (AST) to platelet ratio index (APRI), serum biomarkers (Enhanced Liver Fibrosis (ELF) panel, Fibrometer, FibroTest, or Hepascore), or imaging (transient elastography (TE), MR elastography (MRE), acoustic radiation force impulse imaging, or supersonic shear wave elastography); at least a 5% decrease in circulating levels of cytokeratin-18 fragments relative to circulating levels of cytokeratin-18 fragments before resection; at least a 5% improvement in FIB-4 index, aspartate aminotransferase (AST]) to platelet ratio index (APRI), serum biomarkers (Enhanced Liver Fibrosis (ELF) panel, Fibrometer, FibroTest, or Hepascore), or imaging (transient elastography (TE), MR elastography (MRE), acoustic radiation force impulse imaging, or supersonic shear wave elastography) relative to FIB-4 index, aspartate aminotransferase (AST]) to platelet ratio index (APRI), serum biomarkers (Enhanced Liver Fibrosis (ELF) panel, Fibrometer, FibroTest, or Hepascore), or imaging (transient elastography (TE), MR elastography (MRE), acoustic radiation force impulse imaging, or supersonic shear wave elastography) before resection; at least a 5% decrease in liver stiffness relative to liver stiffness before resection, as measured by vibration controlled transient elastography (VCTE/FibroScan); an improvement in NAS by at least 2 points, with at least 1-point improvement in hepatocellular ballooning and at least 1-point improvement in either lobular inflammation or steatosis score, and no increase in the fibrosis score, relative to NAS, hepatocellular ballooning, lobular inflammation, steatosis, and fibrosis scores before resection; at least a 5% improvement in NFS scores relative to NFS scores before resection; or, at least a 5% improvement in any of the above listed NAFLD parameters as compared to a sham intervention or a placebo.

In various embodiments, resection therapy is provided to achieve the following therapeutic goals or endpoints for patients with diabetes and a first phase of treatment is considered successful for these patients if any one or more of the following therapeutic goals or endpoints is reached: at least one of a 20% decrease in the dose of oral antidiabetic drugs (OAD) or a reduction in the dose of OAD in at least 20% of the patients.

In various embodiments, resection therapy is provided to achieve the following therapeutic goals or endpoints for patients with diabetes and a first phase of treatment is considered successful for these patients if any one or more of the following therapeutic goals or endpoints is reached: at least one of a 20% decrease in the dose of insulin or a reduction in the dose of insulin in at least 20% of the patients or preventing at least 10% of patients from progressing from OAD to insulin therapy.

In various embodiments, resection therapy is provided to achieve the following therapeutic goals or endpoints for patients with diabetes and a first phase of treatment is considered successful for these patients if any one or more of the following therapeutic goals or endpoints is reached: at least one of decreasing the incidence of severe hypoglycemic events by 5% or decreasing the incidence of severe hyperglycemic events in at least 25% of the patients.

If any one of the above therapeutic goals or endpoints is met, therapy is completed, and no further resection is performed. If none of the above therapeutic goals or endpoints are met, then the entire resection procedure and evaluation, less the screening process, can be repeated for a second therapy phase, and subsequent therapy phases. If therapeutic goals or endpoints are still not met, the patient may wait for at least four weeks each time between each resection procedure and each evaluation.

The above examples are merely illustrative of the many applications of the system of present specification. Although only a few embodiments of the present invention have been described herein, it should be understood that the present invention might be embodied in many other specific forms without departing from the spirit or scope of the invention. Therefore, the present examples and embodiments are to be considered as illustrative and not restrictive, and the invention may be modified within the scope of the appended claims.

What is claimed is:

1. A device for resecting a body tissue, comprising:
a cap configured to attach to a distal end of an endoscope wherein the cap comprises a central lumen;
a suction source configured to apply a negative pressure through the central lumen of the cap; and
at least one resection component positioned around an outer surface of the cap, wherein the at least one resection component has an inner surface comprising at least one anchor, wherein the at least one resection component comprises a first material, the at least one anchor comprises a second material, and wherein the first material is different from the second material, wherein the at least one resection component is configured to change from a first configuration when positioned on the cap to a second configuration once deployed, wherein, in the second configuration, the at least one resection component is configured to encircle a portion of said body tissue with the at least one anchor contacting the tissue to secure the resection component to constrict the tissue and reduce blood flow to the tissue.

2. The device of claim 1, wherein the at least one resection component comprises a band, a loop, an O-ring, an elastic circle, or any other component adapted to have a first configuration with a circumference of M when subjected to a pressure and adapted to automatically transition to a second configuration with a circumference of N when the pressure is removed, where N is less than M.

3. The device of claim 1, wherein the anchor comprises a wire of the shape-memory material having at least one first portion extending into the at least one resection component and at least one second portion extending out of and toward a center of the at least one resection component.

4. The device of claim 3, wherein the shape memory material comprises Nitinol.

5. The device of claim 3, wherein, in the second configuration, the at least one second portion of the wire is configured to anchor into the body tissue.

6. The device of claim 3, wherein, in the second configuration, the at least one second portion of the wire is in the shape of a spike and is configured to pierce the body tissue.

7. The device of claim 1, comprising 1 to 10 resection components.

8. The device of claim 1, further comprising a catheter extending between the cap and the suction source.

9. A method for resecting a body tissue in a patient, comprising:
providing a device configured to pass through an endoscope comprising:
a cap positioned on the distal end of the device, wherein the cap comprises a central lumen; and
at least one resection component positioned around an outer surface of the cap, wherein the at least one resection component has an inner surface comprising at least one anchor, wherein the at least one resection component is configured to change from a first configuration when positioned on the cap to a second configuration once deployed off the cap, wherein, in the second configuration, the at least one resection component is configured to encircle a portion of said body tissue, anchor into the tissue using the anchor, and to constrict the tissue and reduce blood flow to the tissue;
inserting the device through the endoscope into the patient and advancing the device such that a distal end of the device is positioned in a gastrointestinal tract of the patient;
activating a suction source to apply a negative pressure through the central lumen of the cap;
drawing a portion of body tissue into the central lumen of the cap, wherein the portion includes at least 1 square centimeters of body tissue;
deploying at least one resection component from the device around a base of the body tissue;
removing the device from the patient;
permitting the at least one resection component to remain around the body tissue and to cut off blood flow to the body tissue.

10. The method of claim 9, wherein the at least one resection component comprises a band, a loop, an O-ring, an elastic circle, or any other component adapted to have a first configuration with a circumference of M when subjected to a pressure and adapted to automatically transition to a second configuration with a circumference of N when the pressure is removed, where N is less than M.

11. The method of claim 9, wherein the anchor comprises a wire of a shape-memory material having at least one first portion extending into the at least one resection component and at least one second portion extending out of and toward a center of the at least one resection component.

12. The method of claim 11, wherein the shape memory material comprises Nitinol.

13. The method of claim 11, wherein, in the second configuration, the at least one second portion of the wire is configured to anchor into the body tissue.

14. The method of claim 11, wherein, in the second configuration, the at least one second portion of the wire is in the shape of a spike and is configured to pierce the body tissue.

15. The method of claim 9, wherein the device comprises 1 to 10 resection components.

16. The method of claim 9, wherein the device further comprises a catheter extending between the cap and the suction source.

17. The method of claim 9, used to treat at least one of excess weight, obesity, an eating disorder, metabolic syndrome, dyslipidemia, diabetes, polycystic ovarian disease, fatty liver disease, non-alcoholic fatty liver disease, or non-alcoholic steatohepatitis disease.

18. The method of claim 9, further comprising determining a therapeutic endpoint after the resection.

19. The method of claim 18, wherein the therapeutic endpoint is at least one of a total body weight of the patient decreases by at least 1% relative to a total body weight of the patient before resection, an excess body weight of the patient decreases by at least 1% relative to an excess body weight of the patient before resection, a total body weight of the patient decreases by at least 1% relative to a total body weight of the patient before resection and a well-being level of the patient does not decrease more than 5% relative to a well-being level of the patient before resection, an excess body weight of the patient decreases by at least 1% relative to an excess body weight of the patient before resection and a well-being level of the patient does not decrease more than 5% relative to a well-being level of the patient before resection, a pre-prandial ghrelin level of the patient decreases by at least 1% relative to a pre-prandial ghrelin level of the patient before resection, a post-prandial ghrelin level of the patient decreases by at least 1% relative to a post-prandial ghrelin level of the patient before resection, or an exercise output of the patient increases by at least 1% relative to an exercise output of the patient before resection.

20. The method of claim 18, wherein the therapeutic endpoint is at least one of a glucagon-like peptide-1 level of the patient increases by at least 1% relative to a glucagon-like peptide-1 level of the patient before resection, a leptin level of the patient increases by at least 1% relative to a leptin level of the patient before resection, the patient's appetite decreases, over a predefined period of time, relative to the patient's appetite before resection, a peptide YY level of the patient increases by at least 1% relative to a peptide YY level of the patient before resection, a lipopolysaccharide level of the patient decreases by at least 1% relative to a lipopolysaccharide level of the patient before resection, a motilin-related peptide level of the patient decreases by at least 1% relative to a motilin-related peptide level of the patient before resection, a cholecystokinin level of the patient increases by at least 1% relative to a cholecystokinin level of the patient before resection, a resting metabolic rate of the patient increases by at least 1% relative to a resting metabolic rate of the patient before resection, a plasma-beta endorphin level of the patient increases by at least 1% relative to a plasma-beta endorphin level of the patient before resection, an HbA1c level of the patient decreases by at least 0.3% relative to an HbA1c level of the patient before resection, a triglyceride level of the patient decreases by at least 1% relative to a triglyceride level of the patient before resection, a total blood cholesterol level of the patient decreases by at least 1% relative to a total blood cholesterol level of the patient before resection, or a glycemia level of the patient decreases by at least 1% relative to a glycemia level of the patient before resection.

21. The method of claim 18, wherein the therapeutic endpoint is a cumulative daily dose of the patient's antidiabetic medications decreases by at least 10% relative to a cumulative daily dose of the patient's antidiabetic medications before resection or 10% of the patients eliminate the use of one or more of their antidiabetic medications being used before resection.

22. The method of claim 18, wherein the therapeutic endpoint is at least one of a 10% decrease in either ALT or AST levels relative to ALT or AST levels before resection, at least a 10% improvement in serum ferritin level or an absolute serum ferritin level of less than 1.5 ULN (upper limit normal) relative to serum ferritin levels before resection, at least a 5% improvement in hepatic steatosis (HS) or less than 5% HS relative to HS levels before resection, as measured on liver biopsy, at least a 5% improvement in HS or less than 5% HS relative to HS levels before resection, as measured by magnetic resonance (MR) imaging, either by spectroscopy or proton density fat fraction, at least a 5% improvement in an NAFLD Fibrosis Score (NFS) relative to an NFS before resection, at least a 5% improvement in an NAFLD Activity Score (NAS) relative to an NAS before resection, at least a 5% improvement in a Steatosis Activity Fibrosis (SAF) score relative to an SAF score before resection, or at least a 5% decrease in a mean annual fibrosis progression rate relative to a mean annual fibrosis progression rate before resection, as measured by histology.
